SEQUENCE LISTING (32 sequences)

SEQ ID NO: 1 - DNA, 630 bp, Homo sapiens
tccgaacatgagacccgtctggtggcaaagctatttaaagactacagcagcgtggtgcggccagtggaagaccaccgccaggtcgtggaggtcaccgtgggcctgcagctgatacagctcatcaatgtggatgaagtaaatcagatcgtgacaaccaatgtgcgtctgaaacagcaatgggtggattacaacctaaaatggaatccagatgactatggcggtgtgaaaaaaattcacattccttcagaaaagatctggcgcccagaccttgttctctataacgatgcagatggtgactttgctattgtcaagttcaccaaagtgctcctgcagtacactggccacatcacgtggacacctccagccatctttaaaagctactgtgagatcatcgtcacccactttccctttgatgaacagaactgcagcatgaagctgggcacctggacctacgacggctctgtcgtggccatcaacccggaaagcgaccagccagacctgagcaacttcatggagagcggggagtgggtgatcaaggagtcccggggctggaagcactccgtgacctattcctgctgccccgacaccccctacctggacatcacctaccacttcgtcatgcagcgcctg

SEQ ID NO: 2 - protein, 210 aa, Homo sapiens
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRL

SEQ ID NO: 3 - DNA, 75 bp, Homo sapiens
ggtgacatggtagatctgccacgccccagctgcgtgactttgggagttcctttgttttctcatctgcaggatgag

SEQ ID NO: 4 - protein, 25 aa, Homo sapiens
GDMVDLPRPSCVTLGVPLFSHLQDE

SEQ ID NO: 5 - DNA, 705 bp, Homo sapiens
tccgaacatgagacccgtctggtggcaaagctatttaaagactacagcagcgtggtgcggccagtggaagaccaccgccaggtcgtggaggtcaccgtgggcctgcagctgatacagctcatcaatgtggatgaagtaaatcagatcgtgacaaccaatgtgcgtctgaaacagggtgacatggtagatctgccacgccccagctgcgtgactttgggagttcctttgttttctcatctgcaggatgagcaatgggtggattacaacctaaaatggaatccagatgactatggcggtgtgaaaaaaattcacattccttcagaaaagatctggcgcccagaccttgttctctataacgatgcagatggtgactttgctattgtcaagttcaccaaagtgctcctgcagtacactggccacatcacgtggacacctccagccatctttaaaagctactgtgagatcatcgtcacccactttccctttgatgaacagaactgcagcatgaagctgggcacctggacctacgacggctctgtcgtggccatcaacccggaaagcgaccagccagacctgagcaacttcatggagagcggggagtgggtgatcaaggagtcccggggctggaagcactccgtgacctattcctgctgccccgacaccccctacctggacatcacctaccacttcgtcatgcagcgcctg

SEQ ID NO: 6 - protein, 235 aa, Homo sapiens
SEHETRLVAKLFKDYSSVVRPVEDHRQVVEVTAGLQLIQLINVDEVNQIVTTNVRLKQGDMVDLPRPSCVTLGVPLFSHLQDEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRL

SEQ ID NO: 7 - DNA, 690 bp, Homo sapiens
tccgaacatgagacccgtctggtggcaaagctatttaaagactacagcagcgtggtgcggccagtggaagaccaccgccaggtcgtggaggtcaccgtgggcctgcagctgatacagctcatcaatgtggatgaagtaaatcagatcgtgacaaccaatgtgcgtctgaaacagggtgacatggtagatctgccacgccccagctgcgtgactttgggagttcctttgttttctcatctgcaggatgagcaatgggtggattacaacctaaaatggaatccagatgactatggcggtgtgaaaaaaattcacattccttcagaaaagatctggcgcccagaccttgttctctataacgatgcagatggtgactttgctattgtcaagttcaccaaagtgctcctgcagtacactggccacatcacgtggacacctccagccatctttaaaagctactgtgagatcatcgtcacccactttccctttgatgaacagaactgcagcatgaagctgggcacctggacctacgacggctctgtcgtggccatcaacccggaaagcgaccagccagacctgagcaacttcatggagagcggggagtgggtgatcaaggagtcccggggctggaagcactccgtgacctattcctgctgccccgacaccccctacctggacatcacctaccacttc

SEQ ID NO: 8 - protein, 230 aa, Homo sapiens
SEHETRLVAKLFKDYSSVVRPVEDHRQVVEVTAGLQLIQLINVDEVNQIVTTNVRLKQGDMVDLPRPSCVTLGVPLFSHLQDEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHF

SEQ ID NO: 9 - DNA, 20 bp, Artificial
  synthetic
ccggatccgaacatgagacc

SEQ ID NO: 10 - DNA, 23 bp, Artificial
  synthetic
cggaattccaggcgctgcatgac

SEQ ID NO: 11 - DNA, 26 bp, Artificial
  synthetic
cggaattctggaggtgtccacgtgat

SEQ ID NO: 12 - DNA, 23 bp, Artificial
  synthetic
ccggatccgccatctttaaaagc

SEQ ID NO: 13 - DNA, 25 bp, Artificial
  synthetic
ggccatgggctccgaacatgagacc

SEQ ID NO: 14 - DNA, 29 bp, Artificial
  synthetic
ccggatcctcaaaagtgrtaggtgatrtc

SEQ ID NO: 15 - DNA, 24 bp, Artificial
  synthetic
cgctatggggctgcttgttgacag

SEQ ID NO: 16 - DNA, 24 bp, Artificial
  synthetic
gacggtatcagtggtctcagtggc

SEQ ID NO: 17 - DNA, 26 bp, Artificial
  synthetic
cagcccagtggaacagggagattcgc

SEQ ID NO: 18 - DNA, 20 bp, Artificial
  synthetic
gatcctcaaattgcagcaca

SEQ ID NO: 19 - DNA, 20 bp, Artificial
  synthetic
agccaaaagatgagaagcca

SEQ ID NO: 20 - DNA, 24 bp, Artificial
  synthetic
tgggagacagctgacggttaaaag

SEQ ID NO: 21 - DNA, 20 bp, Artificial
  synthetic
cgggaatgggaattttacct

SEQ ID NO: 22 - DNA, 20 bp, Artificial
  synthetic
tccagagcagtgatggtgag

SEQ ID NO: 23 - DNA, 24 bp, Artificial
  synthetic
aacatgacaccgcggagactcggg

SEQ ID NO: 24 - DNA, 20 bp, Artificial
  synthetic
aggacttggccttttggagt

SEQ ID NO: 25 - DNA, 20 bp, Artificial
  synthetic
cagtccttggatggtgaggt

SEQ ID NO: 26 - DNA, 24 bp, Artificial
  synthetic
tgatgaggtccgggtgacggtgct

SEQ ID NO: 27 - DNA, 21 bp, Artificial
  synthetic
gtgagagaaaaggcattgctg

SEQ ID NO: 28 - DNA, 21 bp, Artificial
  synthetic
ggttcttgtttgtttctctgc

SEQ ID NO: 29 - DNA, 24 bp, Artificial
  synthetic
ggtgctctctgtcatctccggggt

SEQ ID NO: 30 - DNA, 23 bp, Artificial
  synthetic
gaggcaagcttacttcaatagca

SEQ ID NO: 31 - DNA, 23 bp, Artificial
  synthetic
atgccagtgtttcttgtttcatt

SEQ ID NO: 32 - DNA, 24 bp, Artificial
  synthetic
acacccacgggatcaattatcctc

(19) United States
(12) Patent Application Publication
(10) Pub. No.: US 2007/0269865 A1
(43) Pub. Date: Nov. 22, 2007

(54) RECOMBINANT FRAGMENTS OF THE HUMAN ACETYLCHOLINE RECEPTOR AND THEIR USE FOR TREATMENT OF MYASTHENIA GRAVIS

(75) Inventors: Sara Fuchs, Rehovot (IL); Dora Barchan, Rishon LeZion (IL); Miriam C. Souroujon, Rehovot (IL)

Correspondence Address: BROWDY AND NEIMARK, P.L.L.C.;624 NINTH STREET, NW, SUITE 300, WASHINGTON, DC, 20001-5303, US

(73) Assignee: YEDA RESEARCH AND DEVELOPMENT CO., Ltd., Rehovot (IL)

(21) Appl. No.: 11/777,108

(22) Filed: Jul. 12, 2007

Related U.S. Application Data
(63) Continuation of application No. 09/820,339, filed on Mar. 29, 2001.
Application No. 09/820,339 is a continuation-in-part of application No. 09/423,398, filed on Feb. 18, 2000, now abandoned.

(30) Foreign Application Priority Data
May 7, 1997 (IL) 120792

(51) Int. Cl. C12P 21/04; C07H 21/04; C12N 1/20; C12N 15/00; C12N 5/04; C12N 5/06

(52) U.S. Cl. 435/69.1; 435/252.3; 435/320.1; 435/325; 435/410; 536/23.1

(57) ABSTRACT

Polypeptides capable of modulating the autoimmune response of an individual to human acetylcholine receptor (hAChR), more particularly polypeptides corresponding entirely or partially to the extracellular domain of hAChR α-subunit, are useful in the diagnosis and treatment of myasthenia gravis. Preferred polypeptides are polypeptides corresponding to amino acid residues 1-121 or 122-210 of the hAChR α-subunit sequence, and polypeptides corresponding to amino acid residues 1-121, 1-210 or 1-205 of the hAChR α-subunit sequence in which is inserted, between amino acid residues 58 and 59, a sequence of 25 amino acid residues encoded by the p3A exon of the hAChR α-subunit gene, and fragments, analogs, fused, soluble and denatured forms thereof. DNA molecules encoding said polypeptides are also provided.

### CROSS-REFERENCE TO RELATED APPLICATIONS

The present application is a continuation of application Ser. No. 09/820,339, filed Mar. 29, 2001, which is a continuation-in-part of application Ser. No. 09/423,398, filed Nov. 8, 1999, as a 371 national stage application of PCT/IL98/00211, filed May 6, 1998, the entire contents of all three prior applications are incorporated herein by reference.

### BACKGROUND OF THE INVENTION

1. Field of the Invention

The present invention relates to polypeptides capable of modulating the autoimmune response to acetylcholine receptor, and more particularly to polypeptides corresponding entirely or partially to the extracellular domain of human acetylcholine receptor α-subunit, which polypeptides are useful in the diagnosis and treatment of myasthenia gravis, and to DNA molecules encoding said polypeptides.

ABBREVIATIONS: AChR—acetylcholine receptor; α-BTX—α-bungarotoxin; EAMG—experimental autoimmune myasthenia gravis; GST—glutathione S-transferase; hAChR—human acetylcholine receptor; MG—myasthenia gravis; LNC—lymph node cells; MIR—main immunogenic region.

2. Description of the Related Art

Myasthenia gravis (MG) is a human autoimmune disorder characterized by muscle weakness and fatigability. In this disease, antibodies against the acetylcholine receptor (AChR) bind to the receptor and interfere with the transmission of signals from nerve to muscle at the neuromuscular junction (Patrick and Lindstrom, 1973).

The acetylcholine receptor molecule is a transmembrane glycoprotein consisting of five homologous subunits, organized in a barrel-staves-like structure around a central cation channel, in the stoichiometry of either α2βεδ in fetal, or α2βεδ in mature, muscle Karlin, 1980; Changeux et al., 1984). Noda et al. (1983) described the cloning and sequence analysis of human genomic DNA encoding the α-subunit precursor of muscle acetylcholine receptor, and Schoepfer et al. (1988) reported the cloning of the α-subunit cDNA from the human cell line TE671. Human muscle AChR α-subunit exists in two forms, one of which has 25 additional amino acid residues, inserted between positions 58 and 59, that are coded by the 75 bp exon p3A (Beeson et al., 1990). The α-subunit of AChR contains both the site for acetylcholine binding and the main targets for anti-AChR antibodies.

The autoimmune response in myasthenia gravis is directed mainly towards the extracellular domain of the AchR α-subunit (amino acids 1-210), and within it, primarily towards the main immunogenic region (MIR) encompassing amino acids 61-76 (Tzartos and Lindstrom, 1980; Tzartos et al., 1987; Loutrari et al., 1992).

The involvement of antibodies directed to the MIR and to the ligand binding site of AChR in the autoimmune process can be assessed by the ability of monoclonal antibodies (mAbs) with these specificities to passively transfer experimental autoimmune myasthenia gravis (EAMG) into animals. Examples of such antibodies are mAb 198, mAb 195, mAb 202 and mAb 35 directed towards the MIR of the extracellular portion of hAChR α-subunit (Sophianos and Tzartos, 1989), and mAb 5.5 directed towards the binding site of AChR (Mochly-Rosen and Fuchs, 1981). The anti-MIR antibodies exert their effect by crosslinking AChRs on the muscle surface thereby accelerating their degradation, and the anti-binding site mAbs by blocking and competing with acetylcholine (Souroujon et al., 1986; Asher et al., 1993; Loutrari et al., 1992a). Anti-MIR mAbs have also been shown to accelerate the degradation of AChR in the human cell line TE671 (Loutrari et al., 1992).

MG is currently treated by acetylcholinesterase inhibitors and by non-specific immunosuppressive drugs that have deleterious side effects. It would be preferable to treat MG with a method that involves antigen-specific immunotherapy but leaves the overall immune response intact. One such strategy of specific therapy could involve the administration of derivatives of AChR that do not induce myasthenia but are capable of affecting the immunopathogenic antibodies. However, since the anti-AChR antibody repertoire in myasthenia gravis has been shown to be polyclonal and heterogeneous (Drachman, 1994), the regulation of the disease requires modulation of many antibody specificities.

Previous studies at the laboratory of the present inventors were directed towards modulating the anti-AChR response and EAMG by either denatured derivatives of Torpedo AChR, e.g. the reduced and carboxymethylated derivative, RCM-AChR (Bartfeld and Fuchs, 1978), synthetic peptides corresponding to specific regions of AChR (Souroujon et al., 1992; Souroujon et al., 1993), or mimotopes selected from an epitope library (Balass et al., 1993). The Torpedo RCM-AChR did not induce EAMG in rabbits and was effective in suppressing the disease. However, RCM-AChR did induce EAMG in rats. The experiments carried out with the synthetic peptides and mimotopes were only partially successful in neutralizing MG autoimmune response, probably due to the incorrect folding of the short peptides that were recognized by only a portion of the anti-AChR antibodies.

MG is currently diagnosed by testing for antibodies against AChR by radioimmunoassay wherein the antigen is crude AChR extracted from human muscle or TE671 cells. This test presents some drawbacks, namely the antigen is not readily available and, in addition, the antibody titers detected are not well correlated with disease severity.

Thus, both a safe and effective treatment for MG, as well as a reliable and convenient diagnosis test, are much desired.

Oral tolerance is the phenomenon of systemic, antigen specific, immunological hyporesponsiveness that results from oral administration of antigen (Weiner, 1997). The potential of oral administration of autoantigens or their derivatives for the amelioration of autoimmune diseases was first demonstrated in a model of collagen-induced arthritis in rats that was suppressed by oral administration of type II collagen (Thompson et al., 1986 and Nagler-Anderson et al., 1986). Since then, many groups have demonstrated suppression of autoimmune responses in a variety of animal models, which led to a series of clinical trials in humans suffering from multiple sclerosis (Weiner et al., 1993), rheumatoid arthritis (Trentham et al., 1993), uveitis (Nussenblatt et al., 1996), and type I diabetes (Schatz et al., 1996). Three basic mechanisms have been suggested to contribute to mucosal antigen-driven tolerance: clonal deletion, clonal anergy, and active suppression. These mechanisms are not mutually exclusive and may occur simultaneously to maintain stable tolerance.

Several factors are known to determine the mechanism of oral tolerance. The dose of antigen administered is the primary determinant of which mechanism predominates and may determine the outcome of oral administration of the antigen (Gregerson et al., 1993; Friedman et al., 1994 and Whitacre et al., 1991). Low doses favor active suppression, while high antigen doses favor clonal deletion and clonal anergy. For instance, oral administration of low doses (20 to 2500 μg) of type II collagen has a positive effect on rheumatoid arthritis patients, whereas larger doses did not induce active suppression of the autoimmune process and did not provide protection (Sieper et al., 1996). Similar results were also obtained in a diabetes model in mice (Bergerot et al., 1996).

Even though substantial progress has been made in elucidating the immunological mechanisms associated with antigen-specific oral tolerance, there are still many important aspects to be investigated. These include the delineation of antigen uptake and delivery in the gut, antigen processing and presentation in the gut-associated lymphoid tissue (GALT) and costimulation requirements.

One of the open questions concerns the importance of the chemical nature of the fed tolerogen for the induction of systemic tolerance (Fowler et al., 1997). Orally administered particulate antigens often induce an active immune response, in contrast to the tolerance induced by the same antigens in soluble form (McGhee et al., 1992 and Ermak et al., 1994). The degree of nativity of the antigens is also an important issue. For instance, oral administration of type II collagen in its native form, leads to the induction of chronic autoimmune arthritis in mice, suggesting that the conformation of an orally introduced antigen could be a key factor in induction of systemic tolerance (Terato et al., 1996).

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

It has now been found according to the present invention that polypeptides comprising sequences corresponding to the entire extracellular domain of the human AChR α-subunit, or to fragments thereof, are capable of modulating the autoimmune response to AChR. These polypeptides, herein referred to as “biologically active” polypeptides, were found to affect the antigenic modulation of AChR in TE671 cells in vitro, and to modulate the course of EAMG in vivo; they were effective in suppressing the disease both in EAMG that was passively transferred by monoclonal anti-AChR antibodies, and in EAMG that was actively induced by immunization with AChR, while they did not induce any symptoms of MG in the rat model system; they were further successful in both preventing EAMG and in suppressing an ongoing disease when administered nasally or orally to model rats.

Thus, the present invention provides, in one aspect, a polypeptide capable of modulating the autoimmune response of an individual to acetylcholine receptor, the polypeptide being selected from the group consisting of:

(i) a polypeptide (SEQ ID NO:6) corresponding to amino acid residues 1-210 of the human acetylcholine receptor (hAChR) α-subunit sequence depicted in FIG. 1 (herein “Hα1-210”), in which is inserted, between amino acid residues 58 and 59, a sequence of 25 amino acid residues encoded by the p3A exon of the hAChR α-subunit gene, depicted in FIG. 2 (herein “Hα1-210+p3A”);

(ii) a polypeptide (SEQ ID NO:8) corresponding to amino acid residues 1-205 of the hAChR α-subunit sequence depicted in FIG. 1 (herein “Hα1-205”), in which is inserted, between amino acid residues 58 and 59, a sequence of 25 amino acid residues encoded by the p3A exon of the hAChR α-subunit gene, depicted in FIG. 2 (herein “Hα1-205+p3A”);

(iii) a polypeptide corresponding to amino acid residues 1-121 of the hAChR α-subunit sequence (SEQ ID NO:2) depicted in FIG. 1 (herein “Hα1-121”);

(iv) a polypeptide (residues 1-146 of SEQ ID NO:6) corresponding to amino acid residues 1-121 of the hAChR α-subunit sequence depicted in FIG. 1, in which is inserted, between amino acid residues 58 and 59, a sequence of 25 amino acid residues encoded by the p3A exon of the hAChR α-subunit gene, depicted in FIG. 2 (herein “Hα1-121+p3A”);

(v) a polypeptide corresponding to amino acid residues 122-210 of the hAChR α-subunit sequence (SEQ ID NO:2) depicted in FIG. 1 (herein “Hα122-210”);

(vi) a polypeptide as in (i) to (v) or the polypeptide Hα1-210 (SEQ ID NO:2) in which one or more amino acid residues have been added, deleted or substituted by other amino acid residues in a manner that the resulting polypeptide is capable of modulating the autoimmune response to acetylcholine receptor or suppressing experimental myasthenia gravis in animal models;

(vii) a fragment of a polypeptide as in (i) to (vi), which fragment is capable of modulating the autoimmune response to acetylcholine receptor or suppressing experimental myasthenia gravis in animal models;

(viii) a polypeptide comprising two or more fragments as in (vii) fused together with or without a spacer;

(ix) a polypeptide or a fragment as defined in (i)-(viii) or the polypeptide Hα1-210 (SEQ ID NO:2) fused to an additional polypeptide at its N- and/or C-termini; and

(x) soluble forms, denatured forms, chemical derivatives and salts of a polypeptide or a fragment as defined in (i)-(ix).

Preferred polypeptides according to the present invention are Hα1-121, Hα122-210 and, in particular, Hα1-210+p3A, Hα1-121+p3A, Hα1-205+p3A, optionally fused to an additional polypeptide, e.g., glutathione S-transferase (GST), and Hα1-210 similarly fused.

Preferably a fragment of Hα1-121 comprises at least the amino acid residues 61-76 of the hAChR α-subunit sequence depicted in FIG. 1, and a fragment of Hα122-210 comprises at least the amino acid residues 184-210 of the hAChR α-subunit sequence depicted in FIG. 1.

In another aspect, the invention encompasses a DNA molecule coding for a biologically active polypeptide according to the invention. This DNA molecule may be selected from genomic DNA, cDNA or recombinant DNA or may be synthetically produced.

The present invention also provides a DNA molecule which includes a nucleotide sequence coding for a polypeptide of the invention, the DNA molecule being selected from the group consisting of:

(i) a DNA molecule comprising the sequence (SEQ ID NO:5) of nucleotides 1 to 630, depicted in FIG. 1, in which the sequence of the p3A exon of the hAChR α-subunit gene, depicted in FIG. 2, is inserted between nucleotides 174 and 175;

(ii) a DNA molecule comprising the sequence (SEQ ID NO:7) of nucleotides 1 to 615, depicted in FIG. 1, in which the sequence of the p3A exon of the hAChR α-subunit gene, depicted in FIG. 2, is inserted between nucleotides 174 and 175;

(iii) a DNA molecule comprising the sequence of nucleotides 1 to 363 of SEQ ID NO:1 depicted in FIG. 1;

(iv) a DNA molecule comprising the sequence (SEQ ID NO:5) of nucleotides 1 to 363 depicted in FIG. 1, in which the sequence of the p3A exon of the hAChR α-subunit gene, depicted in FIG. 2, is inserted between nucleotides 174 and 175;

(v) a DNA molecule comprising the sequence of nucleotides 364 to 630 of SEQ ID NO:1 depicted in FIG. 1;

(vi) DNA molecules which are degenerate, as a result of the genetic code, to the DNA sequences of (i) to (v) and which code for a polypeptide coded for by any one of the DNA sequences of (i) to (v);

(vii) a DNA molecule having a coding nucleotide sequence which is at least 70% homologous to any one of the DNA sequences of (i) to (vi) or to the DNA sequence, SEQ ID NO:1, coding for Hα1-210;

(viii) a DNA molecule as in (i) to (v) or the DNA molecule coding for Hα1-210 (amino acid sequence SEQ ID NO:2), in which one or more codons has been added, replaced or deleted in a manner that the polypeptide coded for by the sequence is capable of modulating the autoimmune response to acetylcholine receptor or suppressing experimental myasthenia gravis in animal models;

(ix) a fragment of a DNA molecule as in (i)-(viii) which codes for a polypeptide capable of modulating the autoimmune response to acetylcholine receptor or suppressing experimental myasthenia gravis in animal models;

(x) a DNA molecule comprising two or more fragments of (ix) fused together with or without a spacer, and which codes for a polypeptide capable of modulating the autoimmune response to acetylcholine receptor or suppressing experimental myasthenia gravis in animal models; and

(xi) a DNA molecule comprising a nucleic acid sequence as defined in (i)-(x) or the DNA sequence, SEQ ID NO:1, coding for Hα1-210 fused to additional coding DNA sequences at its 3′ and/or 5′ end.

Preferred DNA molecules according to the invention are those comprising the sequences of nucleotides 1-363 and 364-630 of SEQ ID NO:1, depicted in FIG. 1, coding for Hα1-121 and Hα122-210, respectively, and particularly the sequences of nucleotides 1-630, 1-615 and 1-363, depicted in FIG. 1, in which the sequence of the p3A exon of the hAChR α-subunit gene, depicted in FIG. 2, is inserted between nucleotides 174 and 175, said DNA molecules coding, respectively, for Hα1-210+p3A (SEQ ID NO:6), Hα1-205+p3A (SEQ ID NO:8) and Hα1-121+p3A (residues 1-146 of SEQ ID NO:6) that comprise the additional 25 amino acid residues coded for by the p3A exon of the hAChR α-subunit gene, as well as a DNA molecule coding for Hα1-210 fused to additional coding DNA sequences, e.g., the sequence coding for GST.

Preferably, a fragment of the DNA molecule according to the present invention codes for a polypeptide comprising at least the amino acid residues 61-76 and/or 184-210 of the hAChR α-subunit sequence (SEQ ID NO:2) depicted in FIG. 1.

In still other aspects, the invention provides replicable expression vehicles comprising a DNA molecule of the invention and prokaryotic or eukaryotic host cells transformed therewith.

A further aspect of the invention relates to a process for preparation of the polypeptides of the invention comprising culturing, under conditions promoting expression, host cells transformed by replicable expression vehicles comprising the DNA molecules of the invention, and isolating the expressed polypeptides.

In yet another aspect, the present invention provides pharmaceutical compositions comprising a pharmaceutically acceptable carrier and, as active ingredient, a polypeptide selected from the group consisting of the polypeptides of the invention and a polypeptide comprising the amino acid residues 1-210 of the hAChR α-subunit depicted in FIG. 1 (Hα1-210), soluble forms, denatured forms, salts and chemical derivatives thereof. The polypeptide Hα1-210 was previously described in the literature as a polypeptide which induces myasthenia gravis (Lennon et al., 1991), but the use of this polypeptide for alleviation and/or treatment of myasthenia gravis is herein disclosed for the first time.

In still another aspect, the present invention provides methods for diagnosis and for alleviation and/or treatment of myasthenia gravis using the polypeptides and pharmaceutical compositions of the invention.

### BRIEF DESCRIPTION OF THE DRAWINGS

FIG. 1 depicts the nucleotide sequence (SEQ ID NO:1) and the amino acid sequence coded thereby (SEQ ID NO:2) corresponding to the extracellular domain of the hAChR α-subunit (amino acid residues 1-210).

FIG. 2 depicts the nucleotide sequence (SEQ ID NO:3) and amino acid sequence coded thereby (SEQ ID NO:4) corresponding to the p3A exon of the hAChR α-subunit gene.

FIGS. 3A-C depict Coomassie staining (FIG. 3A) and Western blots with mAb 198 (FIG. 3B) or mAb 5.5 (FIG. 3C) of Hα1-210+p3A, Hα1-210, Hα1-121+3pA, Hα1-121 and Hα122-210 fused to glutathione S-transferase (GST) at the N-terminal (lanes 1 to 5, respectively). GST alone (lane 6) served as a control.

FIG. 4 depicts results of an ELISA assay showing binding of mAb 198 to Hα1-210+p3A (filled squares), Hα1-210 (open squares), Hα1-121+p3A (filled circles) and Hα1-121 (open circles).

FIG. 5 depicts results of an ELISA assay showing binding to Hα1-210+3pA of mAb 198 (filled squares), mAb 5.5 (open triangles), mAb 195 (filled “upside down” triangles), mAb 202 (filled “upright” triangles) and mAb 35 (open circles).

FIG. 6 depicts results of an ELISA assay demonstrating inhibition of mAb198 (0.1 μg/well) binding to AChR by the following polypeptides: Hα1-210+3pA (filled squares), Hα1-210 (open squares), Hα1-121+3pA (filled circles), Hα1-121 (open circles) and GST (filled triangles), at concentrations of 0.05-10 μg/well.

FIG. 7 depicts the inhibition effect of the polypeptides of the invention on AChR degradation induced by mAb 198. TE671 cells were incubated with (a) medium, (b) 1 μg/ml mAb 198, (c-g) 1 μg/ml of mAb 198 preincubated with either Hα1-121 (hatched columns) or with Hα122-210 (dark columns) at concentrations of 10 (c), 25 (d), 50 (e), 100 (f) and 200 (g) μg/ml. Residual AChR was monitored by measuring α-bungarotoxin (α-BTX) binding sites.

FIG. 8 depicts the effect of Hα1-121+p3A on AChR degradation induced by different mAbs. Residual AChR was monitored by measuring α-BTX binding sites. TE671 cells were incubated with medium alone (leftmost column) or with added mAb 198 (1 μg/ml), mAb 35 (1 μg/ml), mAb 195 (5 μg/ml) or mAb 202 (5 μg/ml) either without (dotted columns) or following preincubation of the mAbs with Hμ1-121+p3A (hatched columns).

FIGS. 9A-B depict the effect of nasal administration of Hα1-210+p3A and Hα1-121+p3A on T cell responses to Torpedo AChR (0.25 μg/ml) (FIG. 9A), and IL-2 production in culture (FIG. 9B). Both assays were performed on cells pooled from lymph nodes taken 5 weeks after immunization with AChR from treated and control animals.

FIGS. 10A-B depict the effect of nasal pretreatment on the antibody titers to Hα1-210+p3A (FIG. 10A) and to rat AChR (FIG. 10B), in sera from animals treated with Hα1-210+p3A or control vehicle (GST), at 4 and 8 weeks after immunization with Torpedo AChR

FIGS. 11A-B depict the effect of oral pretreatment with Hα1-210+p3A and Hα1-205+p3A on the mean clinical score of EAMG (FIG. 11A) and on body weight (FIG. 11B).

FIGS. 12A-B depict the effect of oral pretreatment with Hα1-210+p3A and Hα1-205+p3A on T cell responses to Torpedo AChR (0.25 μg/ml) (FIG. 12A), and on the antibody titers to rat AChR (FIG. 12B).

FIGS. 13A-B depict the effect of oral treatment with denatured Hα1-205+p3A on an ongoing EAMG. The mean clinical score (FIG. 13A) and the mean body weight change (FIG. 13B) were monitored for 7 weeks following the beginning of treatment.

FIGS. 14A-C show immunochemical characterization of AChR-derived recombinant on SDS-PAGE and Western blots fragments. Torpedo AChR (5 μg; lane 1) and different recombinant fragments of human AChR α-subunit (20 μg each; GST-Hα1-210, lane 2; Trx-Hα1-210, lane 3 and Hα1-205, lane 4) were resolved on 12% SDS-PAGE and stained by Coommassie blue (FIG. 14A) or blotted to nitrocelluse membranes that were then overlaid with ¹²⁵I-α-BTX (FIG. 14B) or with mAb 198 followed by ¹²⁵I-goat-anti-mouse (FIG. 14C).

FIG. 15 shows a graph of inhibition of mAb 198 binding to Torpedo AChR by different fragments of human AChR. MAb 198 was preincubated in the presence of different concentrations of recombinant fragments and added to microtiter plates coated with Torpedo AChR. Bound mAb 198 was detected by determination of alkaline phosphatase activity.

FIG. 16 shows a graph of the effect of oral treatment with recombinant fragments on ongoing EAMG. Torpedo AChR was injected to induce EAMG and rats were treated twice a week by oral administration of OVA, Trx, Trx-Hα1-210, denTrx-Hα1-210, Hα1-205, or denHα1-205, starting eight days following AChR injection, at the acute phase of EAMG. Treatments were performed as described in Materials and Methods section of Example 2. Representative out of three independent experiments. *P<0.005

FIGS. 17A-B show bar graphs of the effect of oral administration of recombinant fragments on cytokines (FIG. 17A) and costimulatory factors (FIG. 17B). Lymph node cells from rats treated at the acute phase of EAMG with OVA (clear columns), Trx-Hα1-210 (hatched columns) or Hα1-205 (dotted columns) were cultured for 2 days in the presence of AChR, and mRNA was prepared. The mRNA expression level of cytokines or costimulatory factors (and of β-actin as control) was determined by PCR-ELISA and the data are expressed as the relative value compared to the OVA-treated group which was designated 100%. *P<0.005; **P<0.01

FIGS. 18A-B show graphs of the effects of tolerogen conformation on T and B cell proliferation. Proliferation of B and T cells from myasthenic rats in response to Torpedo AChR, Trx-Hα1-210, Hα1-205 and Trx was determined as described in Materials and Methods section of Example 2. The level of B-cell proliferation was determined by alkaline phosphatase activity (FIG. 18A) and proliferation of T-cells was determined by measuring thymidine incorporation (FIG. 18B).

### DETAILED DESCRIPTION OF THE INVENTION

Patients with the neuromuscular disease myasthenia gravis are characterized by the pathogenic autoantibodies, directed towards AChR, that they develop (Aharonov et al., 1975). The α-subunit of AChR appears to be the prime target (major auto antigen) for these pathogenic autoantibodies, and within it especially the extracellular domain. Experimental autoimmune myasthenia gravis (EAMG) is also a T cell-dependent antibody-mediated autoimmune disease of the neuromuscular junction in which AChR is the major autoantigen and which serves as a model for myasthenia gravis.

Human muscle AChR α-subunit exists as two isoforms consisting of 437 and 462 amino acid residues (Beeson et al., 1990). The two isoforms are identical in their amino acid composition except for a sequence of 25 additional amino acid residues inserted after position 58 in the extracellular domain of the longer variant. These additional amino acids are encoded by the 75 bp exon p3A.

According to the present invention, it was found that the polypeptides herein designated Hα1-210, Hα1-210+p3A, Hα1-121, Hα1-121+p3A, Hα1-205+p3A and Hα122-210 are capable of modulating the autoimmune response to AChR and of suppressing experimental myasthenia gravis in animal models.

In order to develop an antigen-specific therapy for oral tolerance, orally or nasally recombinant fragments corresponding to the extracellular domain of the human AChR α-subunit were administered, and successful induction of protection against EAMG and suppression of an already ongoing disease were achieved. These effects on EAMG were shown to be accompanied by reduced AChR-specific cellular and humoral responses (Barchan et al., 1999 and Im et al., 1999). This is different from earlier reports in which Torpedo AChR was used for the induction of mucosal tolerance. In the latter studies, protection against EAMG was accompanied by increased anti-AChR antibody levels, probably due to the high immunogenicity of Torpedo AChR (Drachman, 1996 and Shi et al., 1998).

In order to investigate the role of tolerogen conformation for the induction of oral tolerance in myasthenia gravis, the present inventors used recombinant fragments corresponding to the extracellular domain of the human AChR α-subunit, which differ in their conformation. The different fragments were orally administered to Lewis rats during the acute phase of EAMG and their effects on disease modulation were followed. It was demonstrated that a ‘more native’ fragment, Trx-Hα1-210, which is a fusion of thioredoxin and Hα1-210, failed to induce oral tolerance, whereas a ‘less native’ fragment, Hα1-205, induced tolerance and was efficient in treating ongoing EAMG. This finding was supported by the observation that these two fragments induced different changes in the cytokine profile and in the expression of costimulatory factors.

The present invention relates to the novel polypeptides Ha1-121, Hα1-121+p3A, Hα122-210, Hα1-205+p3A and Hα1-210+p3A as well as to analogs, fragments, fused derivatives (fusion polypeptides), chemical derivatives and salts thereof, and to novel analogs, fragments, fused derivatives (fusion polypeptides), chemical derivatives and salts of the peptide Hα1-210.

Analogs according to the invention are polypeptides in which one or more amino acid residues have been added to, replaced in or deleted from the original polypeptide in a manner that the resulting polypeptide retains its biological activity of suppressing experimental myasthenia gravis in animal models. Preferably, the analog is a variant of the original polypeptide or a biologically active fragment thereof which has an amino acid sequence having at least 70% identity to the “native” amino acid sequence of the original polypeptide and retains the biological activity thereof. More preferably, such a sequence has at least 80% identity, at least 90% identity, or most preferably at least 95% identity to the native sequence. These analogs may be prepared by known synthesis procedures and/or by genetic engineering methods, for example by expressing a DNA molecule modified by site-directed mutagenesis.

The term “sequence identity” as used herein means that the sequences are compared as follows. The sequences are aligned using Version 9 of the Genetic Computing Group's GAP (global alignment program), using the default (BLOSUM62) matrix (values −4 to +11) with a gap open penalty of −12 (for the first null of a gap) and a gap extension penalty of −4 (per each additional consecutive null in the gap). After alignment, percentage identity is calculated by expressing the number of matches as a percentage of the number of amino acids in the claimed sequence.

Analogs in accordance with the present invention may also be determined in accordance with the following procedure. Polypeptides encoded by any nucleic acid, such as DNA or RNA, which hybridize to the complement of the native DNA or RNA under highly stringent or moderately stringent conditions, as long as that polypeptide maintains the biological activity of the native sequence are also considered to be within the scope of the present invention.

Biologically active fragments of the polypeptides of the present invention are also encompassed by the present invention. As long as the fragment is capable of modulating the autoimmune response to acetylcholine receptor and, more particularly, suppressing experimental myasthenia gravis in animal models, any fragment of Hα1-121 or Hα122-210, with or without the p3A, if the p3A site is present in the fragment, are comprehended by the present invention as long as they maintain the capability of suppressing experimental myasthenia gravis in animal models. The preferred such fragments are those which retain amino acid residues 61-76, which is the main immunogenic region (MIR) of the HACHRα subunit. A second preferred class of fragments are those which include amino acid residues 184-210 of the HACHRα subunit sequence which is the acetylcholine binding site of the HACHRα subunit. Also included in the invention are polypeptides containing two or more of such fragments which are fused together with or without a spacer.

Chemical derivatives of the polypeptides of the present invention include modifications of functional groups at side chains of the amino acid residues, or at the N- and/or C-terminal groups. Examples of such derivatives include, but are not limited to, esters of carboxyl and hydroxy groups, amides of carboxyl groups generated by reaction with ammonia or with primary or secondary amines and N-acyl derivatives of free amino groups. Cyclic forms of the polypeptides containing a disulfide bridge between two cysteines residues to stabilize the molecule are also encompassed by the invention. Derivatives which change one amino acid to another are not encompassed by this definition.

The salts of the polypeptides of the invention are pharmaceutically acceptable, i.e., they do not destroy the biological activity of the polypeptide, do not confer toxic properties on compositions containing them and do not induce adverse effects. The term “salts” refers to salts of carboxyl groups as well as to acid addition salts of amino groups of the polypeptide molecule.

A polypeptide of the invention, or a fragment thereof, may be fused to an additional polypeptide at its N- and/or C-termini. For example, recombinant polypeptides were prepared where Hα1-210, Hα1-210+p3A, Hα1-121, Hα1-121+p3A or Hα122-210 were fused to glutathione S-transferase (GST) at the N-terminus, and these molecules were capable of suppressing the immune response to AChR. Other polypeptides may be fused to the N- and/or C-termini of a polypeptide of the invention provided that the fusion does not significantly impair the ability of the polypeptide to suppress experimental myasthemia gravis in animal models.

The results, as presented in Example 2 herein, demonstrate that when an AChR α-subunit extracellular domain polypeptide according to the present invention is fused to another polypeptide (as fusion partner) which causes the AChR α-subunit extracellular domain polypeptide to assume a conformation which is close to its native conformation in the AChR α-subunit, such a fusion causes deleterious effects when administered nasally or orally. The best effect as a tolerogen appears to occur when the polypeptide according to the present invention is allowed to assume a conformation which is farthest from its native conformation. Indeed, from the results in Example 2, it appears that AChR α-subunit extracellular domain polypeptide per se functions best as a tolerogen when it is not fused to any other polypeptide. Thus, if a fusion polypeptide between an AChR α-subunit extracellular domain polypeptide, such as Hα1-120, Hα1-210+p3A, Hα1-121, Hα1-121+p3A, Hα1-205, Hα1-205+p3A, Hα122-210, etc., and another polypeptide is to be encompassed as a polypeptide according to the present invention and is to be used according to the present invention, then such a fusion polypeptide should be first tested to assure that it is not so close to the native conformation of the AChR α-subunit that it will exacerbate rather than ameliorate myasthenia gravis if administered nasally or orally.

There are several ways of testing how close any given fusion polypeptide is to the native conformation of AChR α-subunit extracellular domain. One nonlimiting example of such a test is by binding of the fusion polypeptide to αBTX. The stronger the binding to αBTX, the more likely the AChR α-subunit extracellular domain or fragment thereof in the fusion polypeptide is close to its native conformation. Similarly, another test, in which the strength of binding of monoclonal antibody 198 to the fusion polypeptide is measured, can be used to determine how close the AChR α-subunit extracellular domain, or fragment thereof, in the fusion polypeptide is to its native conformation.

The more weakly the fusion polypeptide binds to αBTX and/or monoclonal antibody 198, the more effective the fusion polypeptide is likely to be as a tolerogen. Subsequent in vivo testing in the EAMG model system can be done to confirm the effectiveness of the tolerogen. Yet another test, which would instead determine fusion polypeptides which should not be used, is whether antibodies are raised when the fusion polypeptide is administered nasally or orally. If antibodies are raised after nasal or oral administration, then the fusion polypeptide is not suitable as a tolerogen.

It will be appreciated by those of skill in the art that the above tests for closeness to native conformation can also be performed on fragments, analogs and chemical derivatives of the AChR α-subunit extracellular domain to determine suitability as a tolerogen for administration to a patient suffering from myasthenia gravis. It is further possible that while a fragment from the AChR α-subunit extracellular domain per se may not be considered suitable as a tolerogen, this same fragment may have its tolerogenicity improved by fusing to another peptide or polypeptide. Thus, if any given fusion polypeptide shows much weaker binding to αBTX and/or monoclonal antibody 198 relative to the fragment from the AChR α-subunit extracellular domain, then such a fusion polypeptide may be a suitable tolerogen and can be further tested for improved effectiveness in the in vivo EAMG model system.

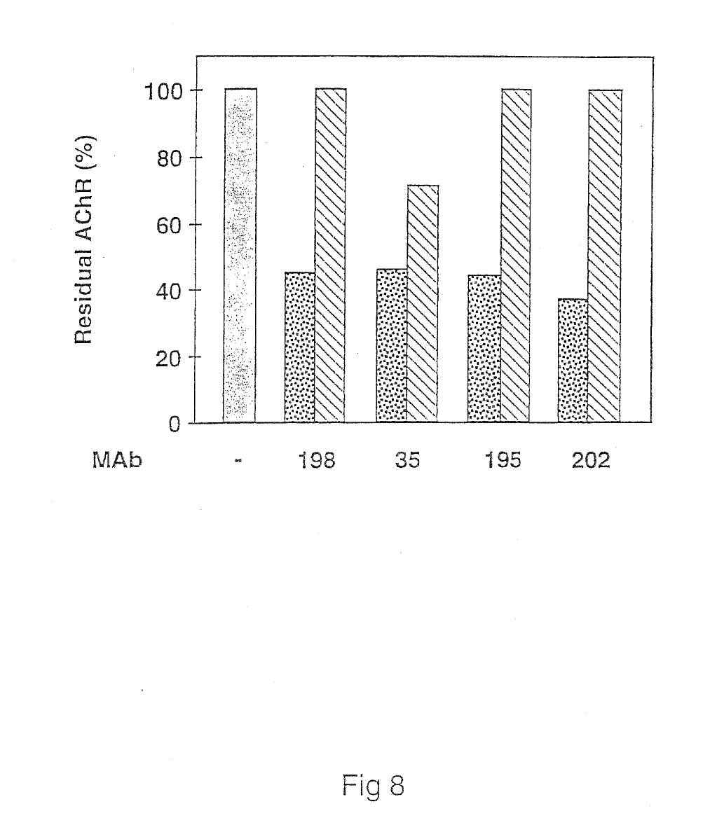

A polypeptide according to the invention corresponding entirely or partially to the extracellular domain of the hAChR α-subunit should be capable of affecting the immunopathogenic response without inducing myasthenia gravis by itself. Since the anti-AChR antibody repertoire in myasthenia gravis has been shown to be polyclonal and heterogeneous (Drachman, 1994), the regulation of myasthenia gravis requires modulation of many antibody specificities. The recombinant polypeptides according to the invention have, indeed, been shown to have a broad specificity as demonstrated by their ability to protect AChR in TE671 cells against antigenic modulation induced by a series of anti-AChR mAbs (FIG. 8) or by polyclonal anti-AChR antibodies from myasthenic rats (data not shown).

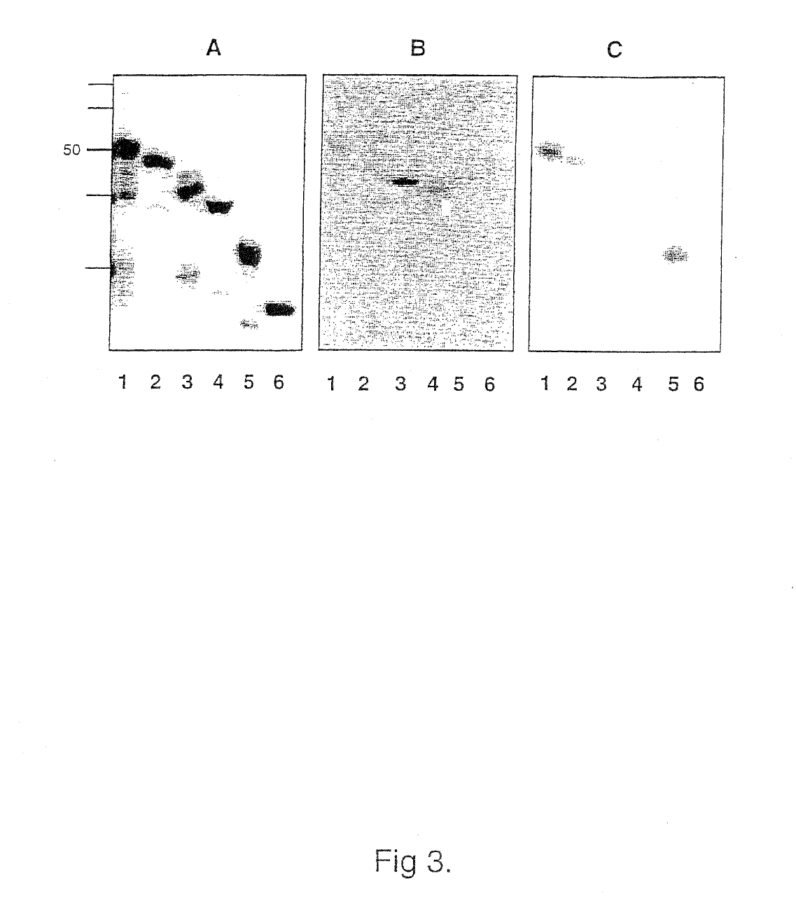

It was shown in several experiments (see FIGS. 3B, 3C, 4 and 6) that the polypeptides comprising the additional 25 amino acid residues coded for by the exon p3A, namely Hα1-121+p3A and Hα1-210+p3A, are more potent in their protective effect in TE671 cells in vitro and in EAMG in vivo. Thus Hα1-121+p3A and Hα1-210+p3A are the most preferred polypeptides according to the invention.

A polypeptide of the invention may be produced by means of recombinant technology or synthetically employing methods well-known in the art.

Recombinant polypeptides according to the invention are prepared by culturing host cells transformed by a suitable expression vector containing a DNA molecule of the invention under conditions promoting expression, and isolating the expressed polypeptide, using standard techniques well known in the art (see, for example, Sambrook et al., 1989; Ausubel et al., 1993).

Soluble forms of the polypeptides that constitute a preferred embodiment of the invention may be generated by suitable chemical modification of natural amino acid residues in the polypeptide, or by substitution of said natural amino acid residues by suitable hydrophilic natural or non-natural amino acids. Alternatively, solubility may be induced by fusion of a polypeptide of the invention to a highly soluble polypeptide partner, such as GST, immunoglobulin or a fragment thereof, maltose binding protein (MBP), thioredoxin or influenza non-structural protein 1 (NS1).

The fused polypeptide of the invention may be used as such, or it may be subjected to further processing in which an active polypeptide of the invention is released. Insertion of a target sequence that is cleavable by specific proteases, such as V8 protease, enterokinase, thrombin or factor Xa, enables the release of the original polypeptide from the recombinant expressed fused polypeptide.

A DNA molecule according to the invention comprises a nucleotide sequence coding for a biologically active polypeptide of the invention. The DNA molecule may be from any origin including non-human sources, and may be selected from genomic DNA, cDNA, recombinant DNA, PCR-produced or synthetically produced DNA.

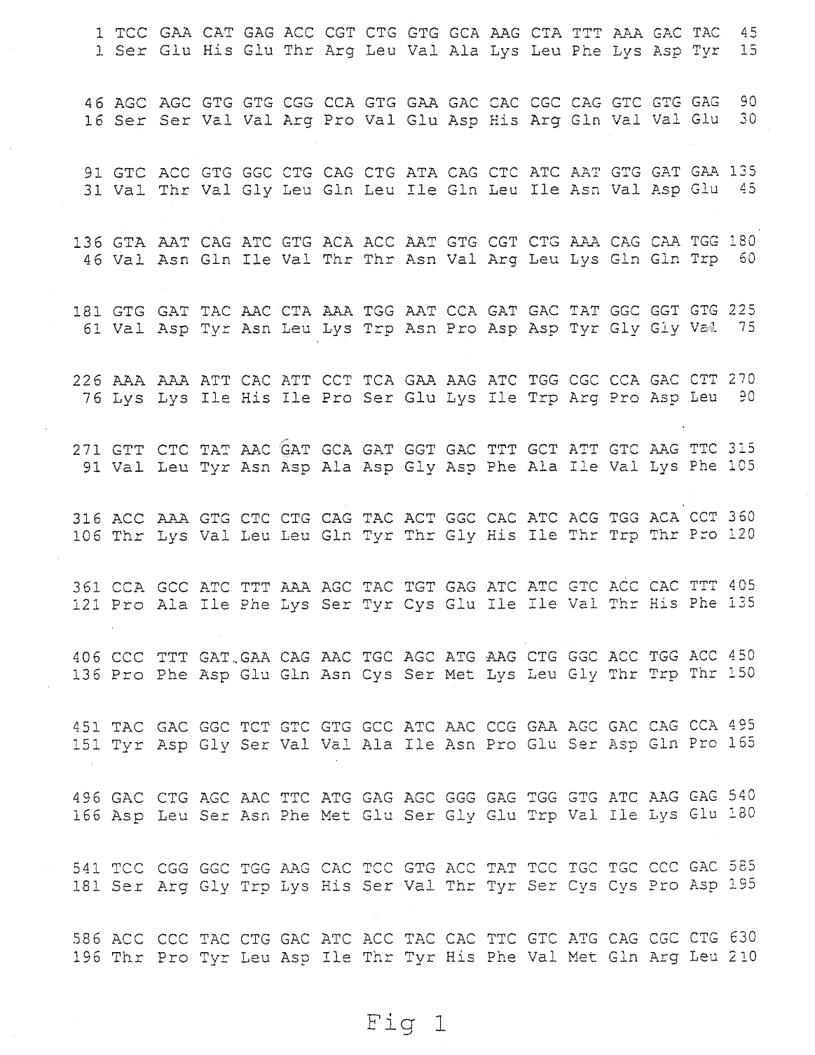

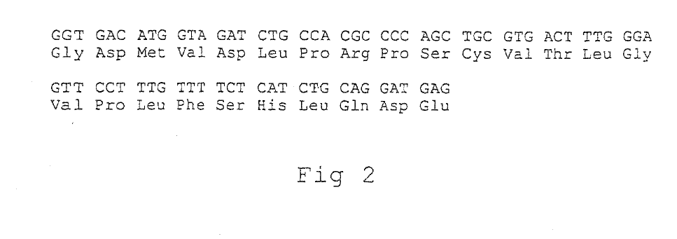

Preferred DNA molecules are those comprising the sequence of nucleotides 1-363 and 364-630 of the hAChR α-subunit (depicted in FIG. 1) coding for Hα1-121 and Hα122-210, respectively, and particularly the sequences of nucleotides 1-630, 1-615 and 1-363 of the hAChR α-subunit in which the sequence of the p3A exon of the hAChR α-subunit gene (depicted in FIG. 2) is inserted between nucleotides 174 and 175, hence coding, respectively, for Hα1-210+p3A, Hα1-205+p3A and Hα1-121+p3A.

A fused DNA molecule according to the invention comprises a nucleic acid sequence coding for a polypeptide of the invention in fusion to additional coding DNA sequences at its 3′ and/or 5′ end. The added DNA sequence may code for a polypeptide endowing the expressed fused polypeptide with favorable characteristics for its purification or for performing its biological activity, i.e., conferring on the original polypeptide molecule a preferred configuration or high solubility.

A DNA molecule of the present invention may be directly isolated from human genomic DNA or cDNA by standard means known in the art involving subcloning genomic or cDNA fractions into a replicable vector, amplifying the subcloned fragments, detecting the relevant clones by their hybridization to the DNA molecules of the present invention or fragments thereof, followed by their isolation, for example as described in Sambrook et al., eds. “Molecular Cloning: A Laboratory Manual”, 2nd ed., Cold Spring Harbor Press, 1989; and in “Current Protocols in Molecular Biology” Current Protocols, Ausubel et al., eds., 1993.

DNA molecules which are at least 70% homologous (sequence identity), preferably 80% homologous, more preferably 90% homologous and most preferably 95% homologous, to H1-210, Hα1-210+p3A, Hα1-205+p3A, Hα1-121, Hα1-121+p3A or Hα122-210 and encoding a polypeptide that has the biological activity of suppressing experimental myasthenia gravis in animal models may be isolated by subjecting a population of cloned genomic DNA or cDNA molecules to hybridization with the above synthesized DNA molecules or fragments thereof under stringent conditions, and isolating the hybridized clones. The term “stringent conditions” refers to hybridization and subsequent washing conditions conventionally referred to in the art as “stringent” (see Sambrook et al., 1989, and Ausubel et al., 1993).

Stringency conditions are a function of the temperature used in the hybridization experiment, the molarity of the monovalent cations and the percentage of formamide in the hybridization solution. To determine the degree of stringency involved with any given set of conditions, one first uses the equation of Meinkoth et al. (1984) for determining the stability of hybrids of 100% identity expressed as melting temperature Tm of the DNA-DNA hybrid:
Tm=81.5° C.+16.6(Log M)+0.41(% GC)−0.61(% form)−500/L
where M is the molarity of monovalent cations, % GC is the percentage of G and C nucleotides in the DNA, % form is the percentage of formamide in the hybridization solution, and L is the length of the hybrid in base pairs. For each 1° C. that the Tm is reduced from that calculated for a 100% identity hybrid, the amount of mismatch permitted is increased by about 1%. Thus, if the Tm used for any given hybridization experiment at the specified salt and formamide concentrations is 10° C. below the Tm calculated for a 100% hybrid according to equation of Meinkoth, hybridization will occur even if there is up to about 10% mismatch.

As used herein, highly stringent conditions are those which are tolerant of up to about 15% sequence divergence, while moderately stringent conditions are those which are tolerant of up to about 20% sequence divergence. Without limitation, examples of highly stringent (12-15° C. below the calculated Tm of the hybrid) and moderately (15-20° C. below the calculated Tm of the hybrid) conditions use a wash solution of 2×SSC (standard saline citrate) and 0.5% SDS at the appropriate temperature below the calculated Tm of the hybrid. The ultimate stringency of the conditions is primarily due to the washing conditions, particularly if the hybridization conditions used are those which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE), 5×Denhardt's reagent, 0.5% SDS, 100 μg/ml denatured, fragmented salmon sperm DNA at a temperature approximately 20°to 25° C. below the Tm. If mixed probes are used, it is preferable to use tetramethyl ammonium chloride (TMAC) instead of SSC (Ausubel, 1993-1998).

Alternatively, a DNA molecule of the invention may be PCR-produced as described, e.g., in Example 1. In general, the PCR-production procedure comprises total RNA purification from relevant cells and generation of first strand cDNA by reverse transcriptase, using either an antisense oligonucleotide mixture or oligo (dT) as a primer. A cDNA fragment may be then amplified in a polymerase chain reaction (PCR) using appropriate sense and antisense primers flanking the target cDNA fragment. The PCR primers may include restriction sites to be used for restriction enzyme digestion followed by cloning into a suitable vector.

Cloning of a DNA molecule of the invention within an appropriate expression vehicle and expression in a suitable host cell enables production and isolation of a biologically active polypeptide or fragment thereof. For this purpose, the DNA molecule is incorporated into a plasmid or viral vector preferably capable of autonomous replication in a recipient host cell of choice. Optionally, the DNA molecule may be cloned into an expression vector in frame with additional coding sequences at its 5′ and/or 3′ end, e.g., the pGEX plasmid vectors that contain GST coding sequences fused upstream to the cloning site. The recombinant expression vector is then used to transform an appropriate prokaryotic or eukaryotic host cell that, under inducing conditions, expresses the polypeptide itself or fused to an additional sequence. In the latter case, insertion of a recognition site for a protease, enables at will the release of the cloned polypeptide from the additional fused polypeptide.

Vectors used in prokaryotic cells include, but are not limited to, plasmids capable of replication in E. coli, for example, pGEX, and bacteriophage vectors such as □gt11, □gt18-23, M13 derived vectors etc.

A vector construct containing the DNA molecule of the invention is then introduced into an appropriate host cell by any of a variety of suitable means known in the art, such as transformation, transfection, lipofection, conjugation, protoplast fusion, electroporation, calcium phosphate precipitation, direct microinjection, etc.

Suitable host cells useful in the invention are prokaryotic cells which include, but are not limited to E. coli, and eukaryotic cells which include, but are not limited to yeast cells such as Saccharomyces cerevisiae, or insect cell lines, for example, Spodoptera frugiperda (Sf9) cells, which are commonly used with the baculovirus expression system, or mammalian cells such as Chinese hamster ovary (CHO) cell lines.

Prokaryotic cells are the preferred hosts in expression systems for producing the polypeptides of the invention. Since non-native polypeptides have been shown to perform better than more native polypeptides, it is expected that polypeptides expressed in prokaryotic systems would perform better than the same polypeptides expressed in eukaryotic systems.

In another aspect, the present invention relates to pharmaceutical compositions comprising a pharmaceutically acceptable carrier and, as active ingredient, a polypeptide selected from polypeptides of the invention, a polypeptide comprising the amino acid residues 1-210 of the hAChR α-subunit depicted in FIG. 1, soluble and denatured forms, salts and chemical derivatives thereof.

The pharmaceutical compositions are for use in the alleviation and/or treatment of myasthenia gravis and may be in any suitable form for administration of polypeptides known in the art, e.g., by injection, inhalation, orally, nasally, etc.

Appropriate pharmaceutically acceptable carriers include physiological carriers, such as water and oils and excipients such as stabilizers and preservative agents. Saline solutions and aqueous dextrose and glycerol solution are suitable for injectable solutions. The active ingredient may also be prepared as a lyophilized dry compound, possibly as a salt, or as a conjugate with a solid carrier/support such as dextran, natural and modified celluloses, etc. The pharmaceutically acceptable carrier of choice will be determined depending on the route the pharmaceutical composition will be administered.

The dosage of the polypeptide and the schedule of the treatment should depend on the route of administration, the patient's condition, age and genetic background and will be determined by a skilled professional person. For example, based on animal studies, it was found that dosage ranges of about 1.4 μq-14 mg and 0.14 μg-0.7 mg/Kg human body weight are suitable for oral and nasal administration, respectively, in humans.

The invention further provides a method for alleviating or treating myasthenia gravis which includes administering to an individual in need thereof an effective amount of a polypeptide in accordance with the present invention.

In contrast to the current methods of treatment of MG using non-specific immunosuppressive drugs, such as steroids, azathioprine or cyclosporine, the method of the present invention is directed to an antigen-specific immunotherapy strategy which suppresses only the adverse autoimmune responses while leaving the overall immune system of the patient intact.

Preferred routes of administration of the polypeptides according to the present invention are the nasal and oral routes.

Nasal tolerization has several advantages as a treatment modality in comparison with oral tolerization: it requires smaller doses of antigen, is more convenient to use and does not require soybean trypsin inhibitor (STI) often used in oral tolerance to inhibit the degradation of the antigen in the gastrointestinal tract. Some successful attempts to modulate experimental autoimmune diseases in animal models by nasal administration of the autoantigen have been recently reported. Thus, Weiner et al. (1994) showed that inhalation of aerosols containing myelin basic protein (MBP) abrogated the clinical symptoms of EAE and significantly reduced the CNS inflammation, DTH reaction and antibody titer to MBP; Dick et al. (1993) reported that nasal administration of retinal extract inhibited the induction of experimental allergic uveitis (EAU) by immunization with this extract; and Ma et al. (1995) demonstrated that nasal administration of the antigen Torpedo AChR diminished the incidence and severity of clinical muscle weakness characteristic of EAMG following immunization with the antigen.

The polypeptides of the present invention are also useful for diagnosis of myasthenia gravis whereby anti-AChR antibodies in the serum of a patient are determined by employing one or more polypeptides of the invention as the test antigen and bound anti-AChR antibody titers indicate the presence of myasthenia gravis. For the diagnostic utility, polypeptides or fusion products closest to the native conformation are preferred.

For the diagnostic test, a serum aliquot from a patient is brought in contact with one or more polypeptides, incubated for about 1 h to overnight at 4°-37° C., followed by the determination of the amount of anti-AChR antibodies bound to the polypeptides by quantitative detection assays known in the art.

In one embodiment, the diagnostic test is to be carried out with immobilized polypeptides in an assay comprising the following steps:

(i) immobilization of one or more polypeptides corresponding entirely or partially to the extracellular domain of human acetylcholine receptor on a suitable solid support;

(ii) incubation of the immobilized one or more polypeptides of step (i) with a serum sample from a patient for 1 h to overnight at 4°-37° C.; and

(iii) determination of the amount of the anti-AChR antibodies bound to the immobilized polypeptides fragments, whereby detection of anti-AChR titers indicates the presence of myasthenia gravis.

The detection of the anti-AChR antibodies may be carried out with labeled anti-human antibodies or labeled Staphylococcus protein A. The label may be a radioactive or fluorescent tag, an enzyme conjugate or another biological recognition tag. Examples of radioactive tags are radioactive isotopes such as ¹²⁵I, ³⁵S, ³²P, ³H, ¹⁴C, etc, which are detected by a scintillation or a γ-counter or by autoradiography. Fluorescent tags are derived from fluorescent compounds such as fluorescein, isothiocyanate, rhodamine, phycoerythrin, phycocyanin, allophycocyanin, o-phthaldehyde and fluorescamine, and are detected by exposure of the bound fluorescent labeled antibody to light of the proper wavelength and monitoring the fluorescence.

Enzyme conjugates useful for detection purposes include, but are not limited to, maleate dehydrogenase, yeast alcohol dehydrogenase, horseradish peroxidase, alkaline phosphatase, beta-galactosidase, catalase and glucose-6-phosphate dehydrogenase. These enzymes are conjugated to the antibody or to protein A and can be monitored by the product they produce when exposed to the appropriate substrate. The chemical moiety thus released can be detected, for example, by chemiluminescence reaction or by spectrophotometry, fluorometry or visual means.

Diagnostic methods based on recognition of biological tags include, for example, coupling of protein A or of the anti-human antibodies to biotin. The biotinylated molecules then can be detected by avidin or streptavidin coupled to a fluorescent compound, to an enzyme such as peroxidase or to a radioactive isotope and the like.

In another embodiment, the diagnostic test is carried out with one or more soluble polypeptides pre-labeled by one of the foregoing labels and tags, whereby anti-AChR antibodies of the patient's serum bound to the polypeptides are separated from the free antigen by precipitation of the antigen-antibody complex by Staphylococcus protein A or anti-human antibodies, and anti-AChR titers are determined as described above.

The diagnostic assays according to the invention have the advantage of avoiding the need to extract the antigen from human tissues or cells, and also provides a more reproducible and safe way for MG detection. The use as antigens of polypeptides that recognize sub-populations of MG-related antibodies further provides a better means for correlating anti-AChR titers with disease severity.

The invention will now be illustrated by the following non-limiting examples and accompanying drawings.

### EXAMPLE 1

### Materials and Methods

i) Monoclonal Antibodies (mAb)

The following monoclonal antibodies were used: mAb directed towards the main immunogenic region (MIR) of the extracellular portion of the hAchR α-subunit (Sophianos and Tzartos, 1989): mAb 198, mAb 195 and mAb 202 elicited in rats against human muscle AChR, and mAb 35 elicited in rats against electric eel AChR, but cross-reacted with AChR from other species, including human; and mAb 5.5 directed towards the binding site of AChR from other species, including human (Mochly-Rosen and Fuchs, 1981), elicited in mouse against Torpedo AChR.

ii) Antibody Binding Assays

Binding of antibodies to AChR or to recombinant polypeptides corresponding entirely or partially to the extracellular domain of the hAChR α-subunit was analyzed by ELISA. Wells of microtiter plates (Maxisorb, Nunc, Neptune, N.J.) were coated by incubation overnight at 4° C. with either Torpedo AChR (1 μg in 100 μl of phosphate-buffered saline (PBS)), or with one of the recombinant polypeptides of the invention (2 μg in 100 μl of 50 mM Tris buffer pH 8.0). Coated plates were washed three times with PBS containing 0.05% Tween-20, then wells were blocked by incubation for 1 h at room temperature (R.T.) with 1% bovine serum albumine (BSA) and 1% hemoglobin in PBS, and the coated blocked plates were then washed and incubated overnight at 4° C. with different amounts of antibody.

For inhibition experiments, each well was coated with 1 μg of Torpedo AChR and a polypeptide of the invention was preincubated with the mAb of choice for 30 min at R.T. before addition to the AChR-coated well. Following a washing step, bound mAb was determined by incubation for 1 h at R.T. with 1:5000 dilution of alkaline phosphatase (AP)-conjugated goat anti-mouse Igs (Jackson ImmunoResearch Labs, Inc., or Biomakor, Ness-Ziona, Israel). The bound antibody was detected by the enzymatic activity of AP using N-para-nitrophenyl-phosphate as a substrate and determining by a microtiter plate reader at 405 nm the color developed after about 40 min.

iii) Determination of AChR Content

AChR content was determined by measuring α-bungarotoxin (α-BTX) binding sites. Tested samples were derived from (a) muscle preparations or from (b) cells grown in a tissue culture.

a) For the muscle preparation, the procedure described by Souroujon et al. (1985) was essentially followed. Briefly, muscle tissue was removed and homogenized in a Sorvall omnimixer for 2 min. at full speed. Two volumes of Tris-HCl buffer, pH 7.5, containing 0.1 M NaCl, 1 mM EDTA, 0.1 mM PMSF and 0.5 mM NaN₃, were used for homogenization. Homogenates were then centrifuged at 48,000×g for 1 h, washed once and recentrifuged as above. The homogenates were stirred overnight at 4° C. in 2 volumes of the above Tris buffer containing Triton X-100 at a final concentration of 1%. The mixture was then centrifuged for 1 h at 100,000×g in a Beckman ultracentrifuge and the recovered supernatant was stored at −70° C. The AChR in the Triton extracts was determined by measuring the amount of ¹²⁵I-α-BTX that coprecipitated with the receptor in ammonium sulfate at 35% saturation. Unbound toxin was removed by filtration through GF/C filters, and radioactivity retained on filters, i.e. toxin bound to receptor, was measured in a γ-counter.

b) For determination of the AChR content in TE671 cells grown in tissue culture, ¹²⁵I-α-BTX (final concentration about 2×10⁻⁹ M; 10⁶ cpm) was added to a confluent cell culture in a 30 mm plate and incubated for 1 h at 37° C. The cells were then washed four times with PBS, released with 1N NaOH and cell-bound radioactivity was evaluated in a γ-counter, after deducting cpm in a control test tube containing an excess of unlabeled α-BTX (final concentration 10⁻⁶ M).

iv) Western Blots

Electrophoresis of recombinant polypeptides corresponding to the entire or partial extracellular domain of the hAChR α-subunit and their blotting were performed essentially as described (Wilson et al., 1985; Neumann et al., 1985). The polypeptides were electrophoresed in 10% polyacrylamide gels and transferred to a nitrocellulose membrane. The membrane was preincubated in PBS containing 0.5% hemoglobin for 1 h at R.T. before addition of 10 μg/ml mAbs and incubation was carried out for additional 3 h at 37° C. The membranes were washed 4 times with PBS, once with PBS containing 0.5% Triton X-100 and then incubated for 1 h at 37° C. with [25]-goat-anti-mouse Ig. After five washes, the blots were exposed to an X-ray sensitive film.

v) Antigenic Modulation in TE671 Cells

Antigenic modulation experiments were performed in 30-mm 12-well plates using TE671 cell cultures. Cells (2×10⁴) were plated in Dulbecco Modified Eagles medium (DMEM) containing 2 mM L-glutamine, 10% fetal calf serum (FCS) and antibiotics (100 U/ml penicillin, 100 μg/ml streptomycin and 250 ng/ml amphotericin B), and grown to confluency for 72 h. The antibodies were added in triplicate to culture wells at a concentration of 1 μg/ml (and for mAbs 195 and 202 also at 5 μg/ml) for 3 h. At the end of the incubation, ¹²⁵I-α-BTX was added at a final concentration of 2×10⁻⁹ M (10⁵ cpm) for an additional hour. AChR content was determined by measuring ¹²⁵I-α-BTX binding, as described in section (iii) above.

In order to test the effect of the polypeptides of the invention on the antigenic modulation induced by the antibodies, the mAbs were preincubated for 1 h at 37° C. with said polypeptides (at concentrations of 10-200 μg/ml, as indicated), before their addition to the cell cultures, and the assay continued as described in section (ii) above.

vi) Passive Transfer of EAMG to Rats.

Lewis female rats (6 weeks old, approximate weight 120 g) were used for passive transfer experiments, as previously described (Asher et al., 1993). For the induction of EAMG, 80 μg of the anti-MIR mAb 198 in 1 ml PBS were injected i.p. into each rat. The tested polypeptide (1 mg) was preincubated with mAb 198 for 30 min at R.T., prior to the injection into rats. The rats were observed for myasthenic symptoms and body weight. At 48 h after the administration of mAb, the animals were sacrificed and their leg muscles were removed for determination of the AChR content according to section (iii) above.

vii) Induction of EAMG and Clinical Evaluation

Animals were injected once in the hind foot pads with 40 μg of Torpedo AChR emulsified in complete Freund adjuvant (CFA) containing 1 mg/rat Mycobacterium Tuberculosis (Difco Lab., Detroit, Mich.). Experimental animals were weighted every week. Clinical EAMG was evaluated as follows: grade 0, no weakness or fatigability; grade 1, weak grip, fatigability; grade 2, weakness, hunched posture at rest, decrease in body weight, tremolousness; grade 3, severe weakness, marked decrease in body weight, moribund; grade 4: dead. Animals were evaluated weekly up to 7-9 weeks after immunization with Torpedo AChR. Blood samples were obtained from the retroorbital plexus.

viii) Lymphocyte Proliferation Assay

Popliteal lymph nodes were aseptically removed and single cell suspensions were prepared in RPMI with 10 mM HEPES. An in vitro T-lymphocyte proliferative assay in response to AChR and the different polypeptides of the invention was performed as follows: Lymph node cells were suspended in RPMI at pH 7.4 containing 10 mM HEPES, supplemented with 2 mM L-glutamine, 1 mM sodium pyruvate, 0.1 mM nonessential amino acids, 5×10⁻⁵ M S-mercaptoethanol and 0.5% normal rat serum, and plated in 96-well flat bottom plates (Corning; 5×10⁵ cells/well). Increasing concentrations of antigen (0.25 to 10 μg/ml of AChR and 10 to 100 μg/ml of a recombinant polypeptide of the invention), were then added to each well. Plates were incubated at 37° C., in 7.5% CO₂ and 90% humidity. Proliferation was assayed after 3 days by measuring incorporation of thymidine-methyl-(3H) into cells. Essentially, the cells were incubated with thymidine-methyl-(³H) (Rotem Ind. Ltd, Beer Sheva, Israel; 0.5 mCi/2.5 ml) for 24 h and then harvested and counted for radioactivity. Results are presented as incorporated cpm following subtraction of cpm in the presence of medium alone.

### Results

Preparation of Recombinant DNA Molecules

DNA molecules encoding the biologically active polypeptides Hα1-210, Hα1-121, Hα122-210, Hα1-205+p3A, Hα1-210+p3A and Hα1-121+p3A were synthesized as follows:

Total RNA was prepared as described (Asher, 1988) from the human TE671 cell line, which expresses the human muscle type nicotinic AChR (Schoepfer et al., 1988). Preparation of cDNA and the polymerase chain reaction (PCR) were performed as described (Barchan et al., 1992). The primers employed to amplify cDNA fragments corresponding to the hAChR α-subunit residue 1-210 (Hα1-210), with or without the p3A exon (Hα1-210+p3A) (Beeson et al., 1990), were constructed with sites that enabled cloning into the fusion protein expression vector pGEX-2T. The primer at the 5′ end, CCGGATCCGAACATGAGACC (SEQ ID NO:9), corresponds to amino acid residues 1-5 of the human AChR α-subunit sequence (nucleotides coding for the first residue are bold), and had a BamHI site (underlined). The primer at the 3′ end had an EcoRI site (underlined) and was complementary to the DNA sequence coding for amino acid residues 206-210, CGGAATTCCAGGCGCTGCATGAC (SEQ ID NO:10).

In a similar way, the shorter clones Hα1-121, Hα1-121+p3A and Hα122-210 were derived by PCR using the above-mentioned Hα1-210 and Hα1-210+p3A clones as templates. For obtaining the two DNA molecules corresponding to amino acid residues 1-121 (with and without the amino acid residues coded by the p3A exon), a primer complementary to the DNA sequence coding for amino acid residues 116-121 with an EcoRI site (underlined) CGGAATTCTGGAGGTGTCCACGTGAT (SEQ ID NO:11), was used at the 3′ end. For the 5′ end, the primer described above corresponding to amino acid residues 1-5 was used. For cloning of the DNA coding for Hα122-210, the primer CCGGATCCGCCATCTTTAAAAGC (SEQ ID NO:12) was used at the 51 end. This primer corresponds to amino acid residues 122-126 (nucleotides coding for residue 122 are in bold) and contains a BamHI site (underlined). The primer used at the 3′ end was the same as described above for the DNA molecule coding for Hα1-210 (complementary to residues 206-210). The PCR amplified DNA sequences were subcloned into the BamHI-EcoRI sites of pGEX-2T expression vector (Pharmacia) (Smith and Johnson, 1988), in frame with the GST-coding DNA sequences at the 5′ end.

The clone Hα1-205+p3A was derived by PCR, using as template the cDNA of hAChR from the TE671 cell line. The primer at the 51 end, GGCCATGGGCTCCGAACATGAGACC (SEQ ID NO:13), corresponded to amino acid residues 1-5 was designed in a way that enabled cloning into a pET8C-derived expression vector by adding a restriction site for NCO I (underlined) the initiation codon ATG. The primer at the 3′ end, CCGGATCCTCAAAAGTGRTAGGTGATRTC (SEQ ID NO:14), where R=A or G, corresponded to the complementary sequence of amino acid residues 200-205, and contained a restriction site for BamHI (underlined) and a stop codon.

All the cloned DNA molecules were sequenced in order to verify their sequence and then used to produce the recombinant polypeptides.

Preparation of Recombinant Polypeptides

The different recombinant DNA molecules subcloned in pGEX-2T plasmid prepared above were used to transform competent E. coli cells (strains JM101 or XL1-blue). The transformed bacteria were grown overnight in LB medium containing ampicillin, then diluted 1:150 in the medium and further grown for additional 3-5 h. Induction of fused polypeptide expression was achieved by adding 0.5 mM IPTG (isopropyl β-D-thiogalactopyranoside) for 2 h. After expression, the bacterial suspension was centrifuged, cells were lysed by freezing and thawing the pellet and resuspended in PBS (10 ml). The preparation was sonicated for five 15-sec periods, and centrifuged for 15 min at 27,000×g. The expressed recombinant fused polypeptides were localized in the precipitate, probably in inclusion bodies. The fused polypeptides were solubilized in 1 ml of 9 M urea, the non-soluble fraction was removed by centrifugation for 45 min at 27,000×g, and the supernatant was diluted in 10 ml of 50 mM Tris buffer, pH 8.0 and dialyzed against the same buffer for 48 h with several changes. After ultracentrifugation for 30 min at 100,000×g, the supernatant was divided into aliquots for storage at −80° C. The protein concentration, determined by the Lowry method, was 1-3 mg/ml, with a yield of 20-50 mg of total protein from one liter of bacterial suspension. The GST-fused polypeptides were isolated using a substrate affinity column according to Smith and Johnson, 1988. A Coomassie brilliant blue staining of the expressed GST-fused polypeptides run on 10% polyacrylamide gel is shown in FIG. 3A: from left to right, lanes 1-6, Hα1-210+p3A, Hα1-210, Hα1-121+p3A, Hα1-121, Hα122-210 and GST, appearing to have MW of 52.5, 50.0, 43.7, 41.2, 37.8 and 29.0 kD, respectively, in agreement with the expected MW calculated based on the encoded amino acid sequences of these polypeptides (see FIG. 1 and FIG. 2).

Expression of Hα1-205+p3A in the pET8C expression system was performed in a similar procedure using E. coli BL21 strain.

Immunochemical Characterization of the Recombinant Polypeptides

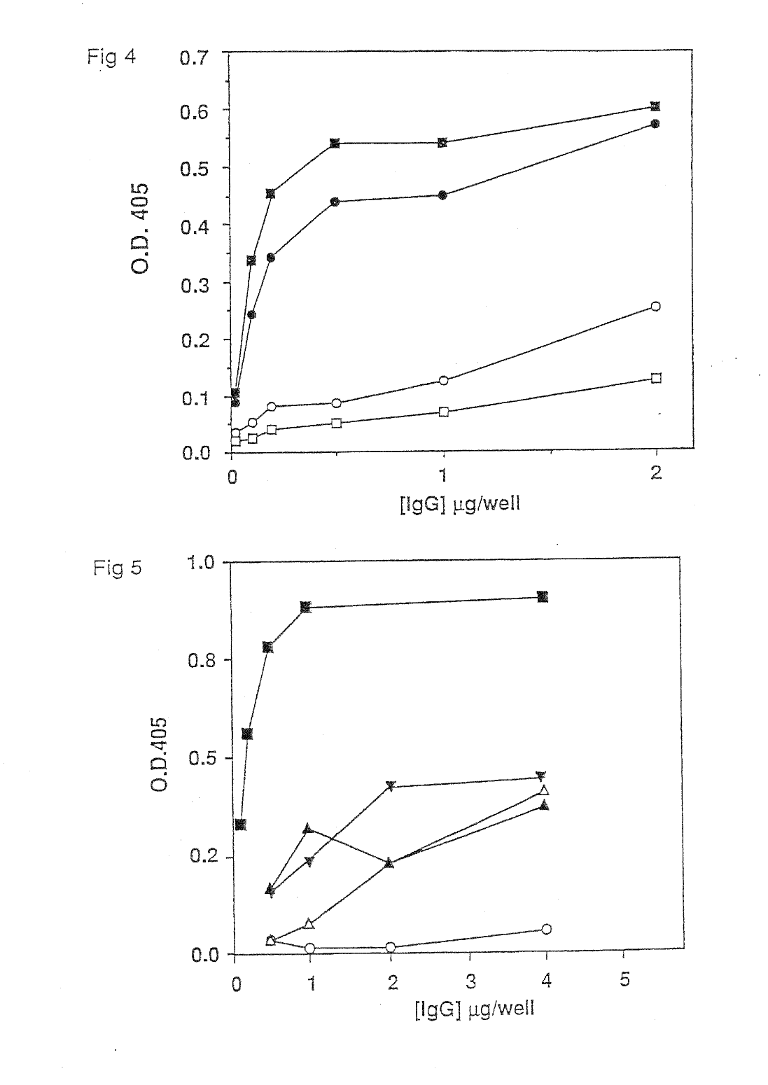

The prepared recombinant polypeptides of were further characterized by their binding to various anti-AChR mAbs as assayed by both Western blots (FIG. 3B—mAb 198; FIG. 3C—mAb 5.5) and by ELISA (FIG. 4 and FIG. 5).

The recombinant polypeptides (20 μg each) were electrophoresed, blotted onto nitrocellulose membrane, and incubated with different mAbs as described in the Materials and Methods section (iv). FIG. 3B shows that mAb 198, which is directed to the MIR, bound to the polypeptide corresponding to the entire extracellular portion of the hAChR α-subunit (Hα1-210) and to its shorter derivative (Hα1-121), that contains the MIR, as well as to their variants including the additional p3A encoded sequence Hα1-210+p3A and Hα1-121+p3A. As expected, mAb 198 did not bind to Hα122-210, which does not include MIR, or to the GST protein itself.

The mAb 5.5, which is directed to the binding site of AChR (Mochly-Rosen and Fuchs, 1981), bound to Hα1-210, Hα1-210+p3A and to Hα122-210, all including the binding site, but it did not bind to Hα1-121, Hα1-121+p3A nor to the GST protein (FIG. 3C). As shown, both mAb 198 and mAb 5.5 bound better to the variants containing the sequence encoded by the p3A exon.

The binding of mAb 198 to the polypeptides of the invention was also determined in ELISA carried out as described in Materials and Methods section (ii), and the results are shown in FIG. 4. In this assay, as in the Western blot, mAb 198 bound better to the polypeptides Hα1-210+p3A and Hα1-121+p3A (filled symbols). Therefore, these longer variants were used in further studies. Three other anti-MIR mAbs (mAb 195, mAb 202 and mAb 35) bound to a lesser extent than mAb 198 to all tested polypeptides (not shown).

FIG. 5 illustrates the binding of various mAbs to Hα1-210+p3A: mAb 198 (filled squares) showed a very strong binding. MAb 35, which is directed against the MIR and is known to depend on the native conformation of AChR, showed very low binding to the tested polypeptides of the invention (open circles). MAb 5.5 which also depends on the native conformation of AChR, bound well to the tested polypeptides in Western blots (FIG. 3C), but to a much lesser extent than mAb 198 in ELISA (open triangles). This poor binding of mAbs 35 and 5.5 may indicate that when bound to ELISA plates only a small fraction of the recombinant polypeptide is properly folded.

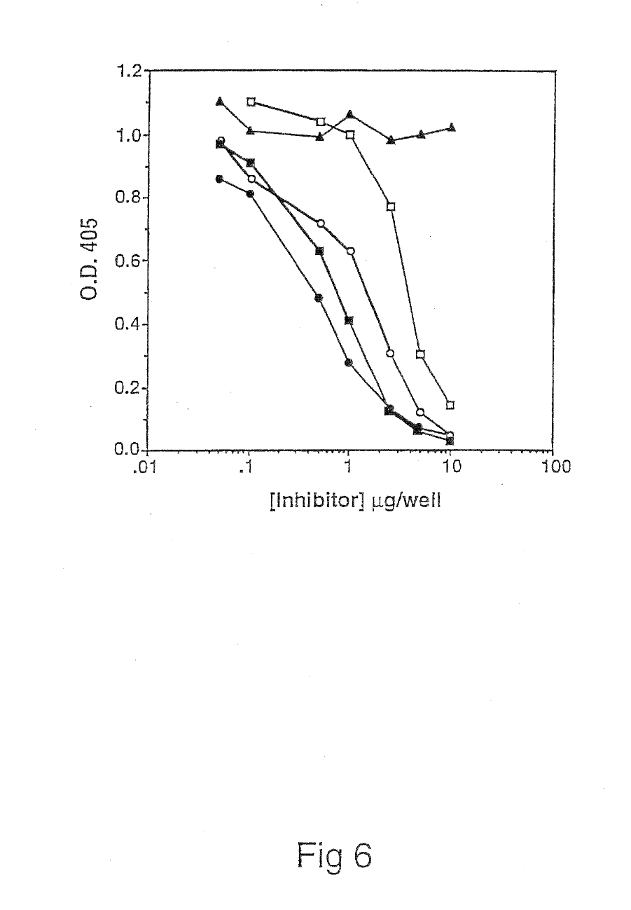

Based on the results of the binding experiments in ELISA, the next step was to test whether the polypeptides of the invention bind to the mAbs also in solution. For that, the ability of the various recombinant polypeptides to inhibit the binding of mAb 198 to Torpedo AChR was tested in ELISA. As shown in FIG. 6, Hα1-210+p3A (filled squares) and Hα1-121+p3A (filled circles) inhibited this binding, with IC₅₀ values of 1.8×10⁻⁷M and 1×10⁻⁷M, respectively, whereas the GST protein (filled triangles) did not, indicating that the solubilized recombinant fused polypeptides may indeed bind to mAb 198 also in solution. As shown above (FIGS. 3B and 4), the variants containing the additional 25 amino acid residues encoded by the p3A exon were more potent in inhibiting mAb 198 binding to AChR than their counterparts lacking this 25-mer.

Effect of the Polypeptides on Antigenic Modulation of AChR in TE671 Cells

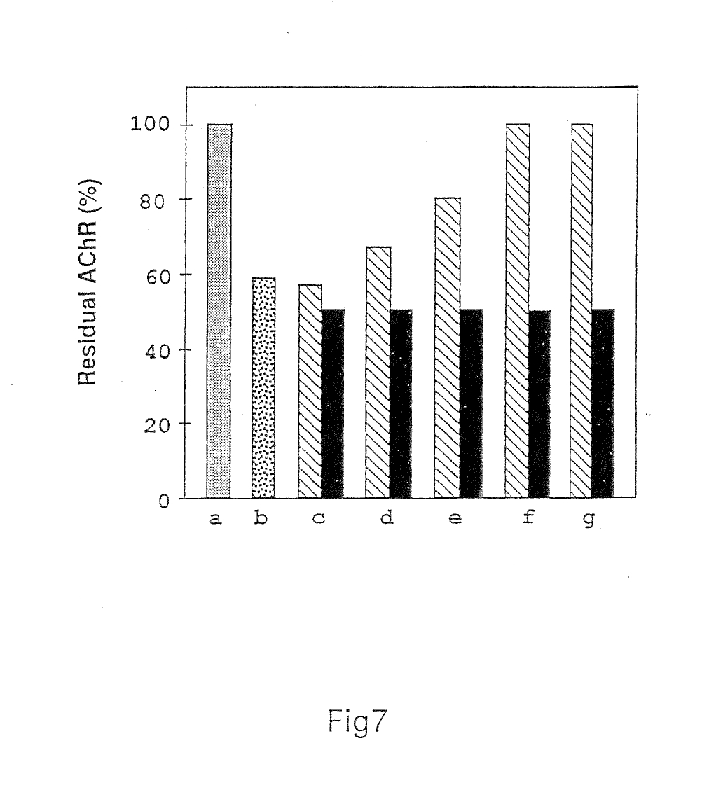

Muscle AChR loss in myasthenia gravis is caused by accelerated degradation of the receptor, brought about by anti-AChR antibodies, a great portion of which are directed to the MIR. This activity of the antibodies can be demonstrated in vitro in cell cultures such as the human cell line TE671. This human medulloblastoma-derived cell line expresses a functional AChR which binds α-BTX and has the α-subunit of the muscle-type AChR. The ability of the recombinant polypeptides Hα1-210 and Hα1-121 to protect the AChR on TE671 cells against accelerated degradation of AChR induced by specific anti-AChR α-subunit mAbs, was examined as follows: Anti-MIR mAbs were preincubated for 1 h at 37° C. with several concentrations of the recombinant polypeptide and then added to the cells. As a control, the mAbs were preincubated with GST or with the Hα122-210 polypeptide that does not include the MIR. The inhibition effect of Hα1-121 on AChR degradation induced by mAb 198 measured as residual α-BTX binding sites, is illustrated in FIG. 7. MAb 198 causes a reduction of 41% in residual AChR following 3 h incubation with the cells (FIG. 7, lane b). Preincubation with increasing concentrations of Hα1-121 had a dose dependent protection effect against the degradation induced by mAb 198 (FIG. 7, c-g, hatched columns). At a concentration of 100 μg/ml of Hα1-121 the TE671 cells were completely protected against the accelerated AChR degradation by mAb 198. Preincubation of mAb 198 with Hα122-210, which does not contain the MIR, did not affect the antigenic modulation induced by mAb 198 and did not block AChR degradation (FIG. 7, c-g, dark columns). Hα1-210, corresponding to the entire extracellular α-subunit domain, had the same effect as the shorter fragmeαnt Hα1-121 (data not shown).

Results of a comparable experiment carried out with other anti-AChR mAbs are shown in FIG. 8. The polypeptide Hα1-121 had a similar protection effect against AChR degradation induced by two other anti-MIR mAbs, mAb 195 and mAb 202, but had a much smaller effect on mAb 35-induced AChR degradation, possibly because of the weak binding of this antibody to Hα1-121 in solution (see FIG. 5).

Modulation by the Polypeptides of EAMG Passively Transferred by mAb 198

The effect of the polypeptides of the invention was also examined in vivo in a well-established animal model disease for myasthenia gravis, designated experimental autoimmune myasthenia gravis (EAMG) (Lindstrom et al., 1976 and 1976a). In animals such as rabbits, mice, guinea-pigs, monkeys and rats, EAMG can be either passively transferred by anti-AChR antibodies, or actively induced by AChR. In both cases, the treated animals show chronic symptoms of the MG disease, i.e. show general weakness, have a hunched posture, develop a flaccid paralysis of the hind limbs, have difficulties in breathing, in swallowing and in reaching food and water supplied to them, all of which result in weight loss. The animals die from respiratory insufficiency, malnutrition and dehydration. In rats, two distinct episodes of weakness occur, especially after immunization with Torpedo AChR in combination with Mycobacterium tuberculosis (killed) H37 Ra, with an acute phase starting 8-10 days after immunization and a chronic phase starting 3-5 weeks later.

TABLE 1
Recombinant fragments modulate experimental myasthenia
passively transferred by a monoclonal anti-AChR antibody

| | | | AchR content* | |
|---|---|---|---|---|
| | Anti-AChR | Myasthenic | Fmoles/ | % of |
| Treatment | mAb 198 | symptoms | mg prot. | control |
| — | − | − | 39.9 ± 6.3 | 100 |
| — | + | + | 19.2 ± 3.5 | 48 |
| Hα1-121 | + | − | 38.8 ± 6.9 | 97 |
| Hα122-210 | + | + | 24.5 ± 2.4 | 61 |
| GST | + | + | 19.2 ± 4.5 | 48 |
| BSA | + | + | 21.4 ± 2.4 | 53 |

*Muscle AChR content was determined by α-bungarotoxin binding to AChR present in Triton X-100 extracts from rat leg muscles, 48 h after Ig administration
The values (mean ± SEM) are averages derived from at least three different animals.

EAMG was passively transferred in rats by mAb 198. The disease was induced within 24-48 h following administration of the antibody (Asher et al., 1993). Muscle AChR content was determined by α-bungarotoxin binding to AChR present in Triton X-100 extracts from rat leg muscles, 48 h after the mAb administration. As previously reported, the myasthenic symptoms were accompanied by a marked reduction in the muscle AChR content (48% of normal control; Table 1). In order to examine the effect of the polypeptides of the invention on the disease symptoms, mAb 198 was preincubated with a 30 fold molar excess of recombinant polypeptides of the invention, or with either GST or BSA as controls, prior to its injection into rats.

As shown in Table 1, the muscle AChR content in the EAMG-induced rats was reduced to 48% of AChR content of control untreated rats. The recombinant polypeptides of the invention were able to modulate in vivo muscle AChR loss and to decrease significantly clinical symptoms of EAMG. It was shown that preincubation of mAb 198 with Hα1-121+p3A prior to its injection into rats, prevented the appearance of myasthenic symptoms. The protected rats had a normal muscle AChR content (97% of control). Similar results were obtained with the Hα1-210+p3A polypeptide (data not shown). On the other hand, preincubation with either Hα122-210+p3A or with GST or BSA did not affect the muscle AChR content significantly (61, 48 and 53% of control, respectively) and did not prevent myasthenic symptoms. Administration of Hα1-121+p3A and Hα122-210+p3A alone did not induce any myasthenic symptoms in rats.

Interestingly, similar protection effect by Hα1-121+p3A and Hα1-210+p3A was demonstrated when the recombinant polypeptide was injected together with mAb 198 without preincubation, or even two hours after the administration of mAb 198 (data not shown).

Protective Effects of Nasal Administration of the Polypeptides of the Invention on Actively Induced EAMG in Rats

Hα1-210+p3A, Hα1-121+p3A and Hα122-210 fused with GST were expressed and solubilized as described above in the preparation of recombinant polypeptides. Nasal tolerance was induced in rats by administration of a daily dose of 2.5 μg of each of said fused polypeptides in 30 μl PBS into each rat nostril, over a period of ten consecutive days. Three days later the rats were immunized with Torpedo AChR (40 μg/rat) injected into the footpads, in Complete Freund's Adjuvant supplemented with 1 mg of Mycobacterium tuberculosis H₃₇RA (DIFCO). Control rats received GST instead of the recombinant polypeptide. Clinical symptoms of EAMG disease, as well as body weight, were monitored weekly. The results of the experiment are summarized in Table 2, showing that all three tested polypeptides had a protective effect in the rats.

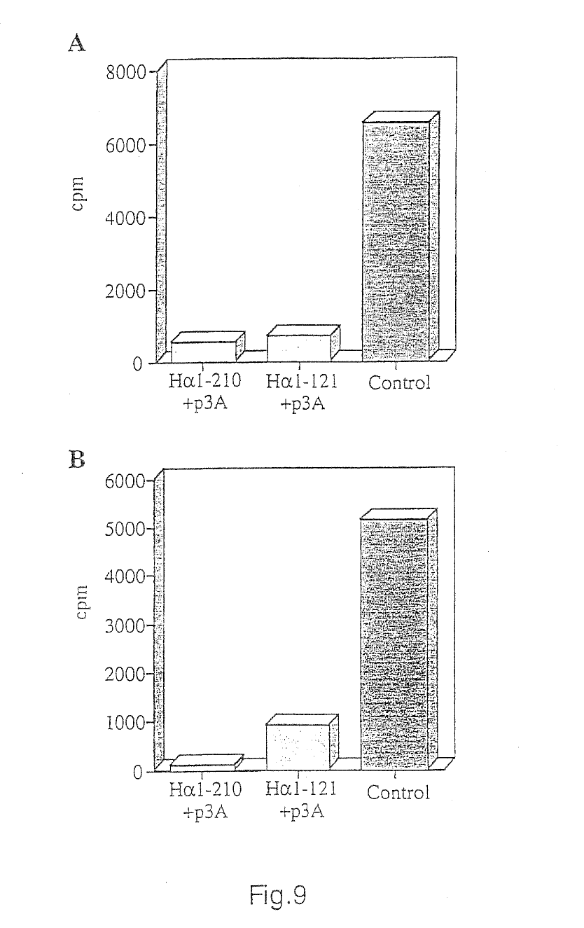

Rats treated intranasally with either of the three recombinant fragments, before immunization with Torpedo AChR, were protected against EAMG, as assessed by clinical symptoms of EAMG as well as by weight loss and muscle AChR content as summarized in Table 2.67%, 56% and 34% of the rats pretreated with Hα1-210+p3A, Hα1-121+p3A and Hα122-210+p3A, respectively, were completely protected and did not develop clinical symptoms of EAMG, and the other rats in these groups were partially protected and had milder symptoms. On the other hand, all rats in the control, GST-pretreated group, were sick. As shown in Table 2, there was a marked effect of the treatment on the weight of the rats. Whereas rats in the control, GST-treated group, exhibited a notable decrease in body weight (12.8±9.2 g) characteristic to EAMG, between 3 weeks and 7 weeks following AChR injection, rats in groups pretreated with AChR fragments increased significantly in their body weight. The protective effect of the nasal treatment was also evident from the receptor content data. As seen in Table 2, there was a decrease of about 55% in AChR content in the control-GST treated rats, and only 11% decrease in AChR content in rats pretreated with Hα1-210+p3A. The recombinant fragments themselves had no myasthenogenic effects under the conditions employed for treatment. Protection against EAMG by nasal administration of the polypeptides of the invention was accompanied by a reduction in the proliferative T-cell response and IL-2 production in response to AChR (FIGS. 9A-B), and in the antibody titers to both Hα1-210+p3A and to self, rat AChR (FIGS. 10A-B).

TABLE 2
The effect of intranasal treatment with human recombinant AChR fragments on EAMG in rats.

| | Clinical score^{a} | | | | | | Δweight | AChR content | |
|---|---|---|---|---|---|---|---|---|---|
| | 0 | 1 | 2 | 3 | 4 | Healthy | 3 w to 7 w | fmoles/mg | |
| Treatment | No/total | | | | | rats % | gr | prot. | % |
| Control | 0/10 | 2/10 | 2/10 | 4/10 | 2/10 | 0 | −12.8 ± 9.2 | 17.5 ± 4.1 | 44 |
| vehicle | | | | | | | | | |
| (GST) | | | | | | | | | |
| Hα122-210 | 3/9 | 3/9 | 1/9 | 1/9 | 1/9 | 33 | +9.2 ± 8.6 | 14.9 ± 1.7 | 38 |
| Hα1-121 + p3A | 5/9 | 1/9 | 2/9 | 0/9 | 1/9 | 56 | +13.6 ± 2.5 | 29.6 ± 4.5 | 75 |
| Hα1-210 + p3A | 6/9 | 1/9 | 1/9 | 0/9 | 1/9 | 67 | +15.0 ± 6.3 | 35.0 ± 3.4 | 89 |
| Normal rats | | | | | | | | 39.5 ± 2.5 | 100 |

^{a}Evaluated 7 weeks after the induction of EAMG.

Suppressive Effects of Nasal Administration of the Polypeptides of the Invention on an Ongoing EAMG.

In order to evaluate the potential of the polypeptides of the invention to affect an ongoing disease, nasal administration of Hα1-210+p3A was initiated 7 days after the induction of EAMG by immunization with Torpedo AChR. At this time rats are known to be at the first, acute phase of EAMG. Other than the time of initiation, the protocol for the nasal administration was as in the previous section on protective effects.

As summarized in Table 3, suppression of EAMG was observed also when nasal treatment with Hα1-210+p3A was initiated after the induction of EAMG. Among the rats treated intranasally with Hα1-21+p3A, 30% were disease-free for at least 8 weeks following induction of EAMG, and in the other rats in the group the symptoms seemed to be milder. There was also an effect of the nasal treatment on the receptor content. As seen in Table 3, there was a loss of 68% in the AChR content in the control (treated intranasally with ovalbumin) rats, and only a 20% loss in the rats treated intranasally with Hα1-210+p3A.

TABLE 3
The effect of intranasal treatment with human recombinant AChR fragment
Hα1-210 + p3A on ongoing EAMG in rats

| | Clinical score^{b} | | | | | | AChR Content | |
|---|---|---|---|---|---|---|---|---|
| | 0 | 1 | 2 | 3 | 4 | Healthy | fmoles/mg | |
| Treatment^{a} | No/total | | | | | Rats % | protein | % |
| Control vehicle (OVA) | 0/10 | 2/10 | 4/10 | 3/10 | 1/10 | 0 | 11 ± 1 | 32 |
| Hα1-210 + p3A | 3/10 | 3/10 | 3/10 | | 1/10 | 30 | 27 ± 8.5 | 80 |
| Normal rats | | | | | | | 34 ± 8.5 | 100 |

^{a}Nasal administration was initiated 7 days after induction of EAMG by immunization with AChR and was continued for 12 consecutive days.
^{b}Clinical evaluation was made 10 weeks after induction of EAMG.

Effects of Oral Administration of the Polypeptides of the Invention on EAMG in Rats.

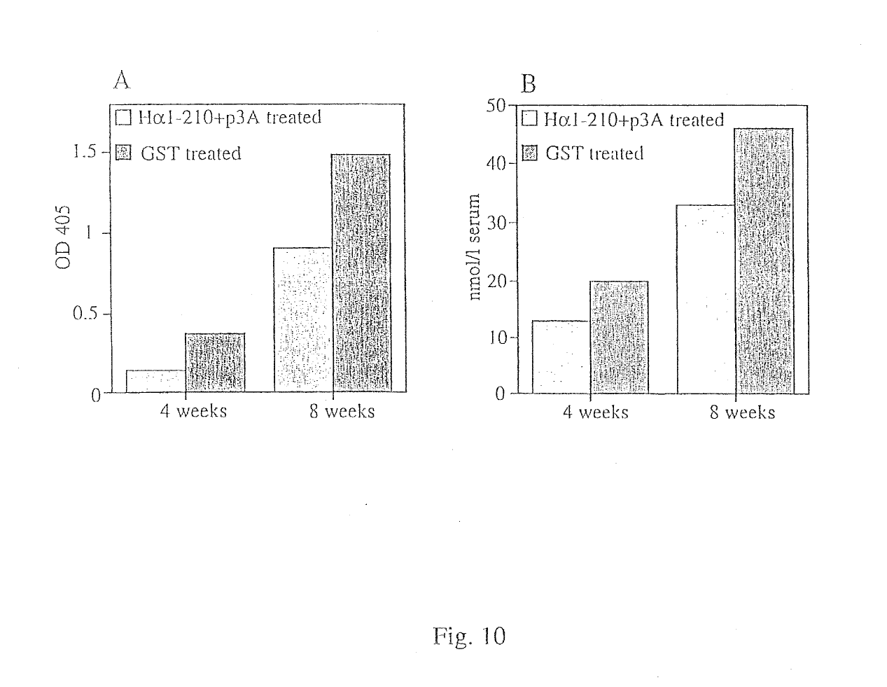

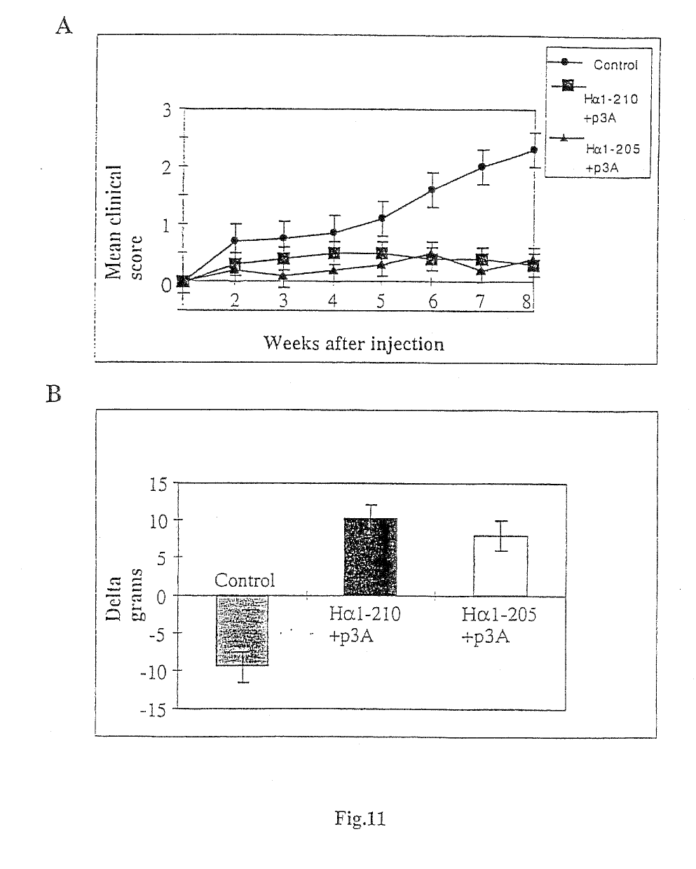

The potential of oral administration of the polypeptides of the invention to prevent EAMG was first investigated. Two recombinant preparations of the extracellular domain of human AChR α-subunit were employed for oral tolerization: Hα1-210+p3A (fused with GST), and the extracellular domain itself (Hα1-205+p3A) expressed in the pET8C expression system with no fusion protein. Rats were fed 5 times with three days interval, each time with 0.6 mg of the recombinant fragment per rat, and AChR was injected in CFA to induce EAMG, three days after the last feeding. Rats were followed clinically, as well as for weight loss for 8 weeks after EAMG induction. As shown in FIG. 11, oral feeding with either GST-fused Hα1-210+p3A or with Hα1-205+p3A had a significant protective effect on the clinical symptoms of EAMG for at least 8 weeks. The values represent the average clinical score in the group at each time point. About 70% of the rats that were pretreated orally, did not develop any clinical symptoms and the other rats in this group were partially protected. The weight of the animals corroborated with the clinical evaluation. Control, nontreated rats, lost about 10 g per rat between 4 and 8 weeks after EAMG induction, whereas rats pretreated orally with the recombinant fragments gained about 10 g per rat during this time interval (FIG. 11). T-cell response to AChR as well as anti-rat AChR antibody titers were also reduced following oral treatment (FIG. 12).

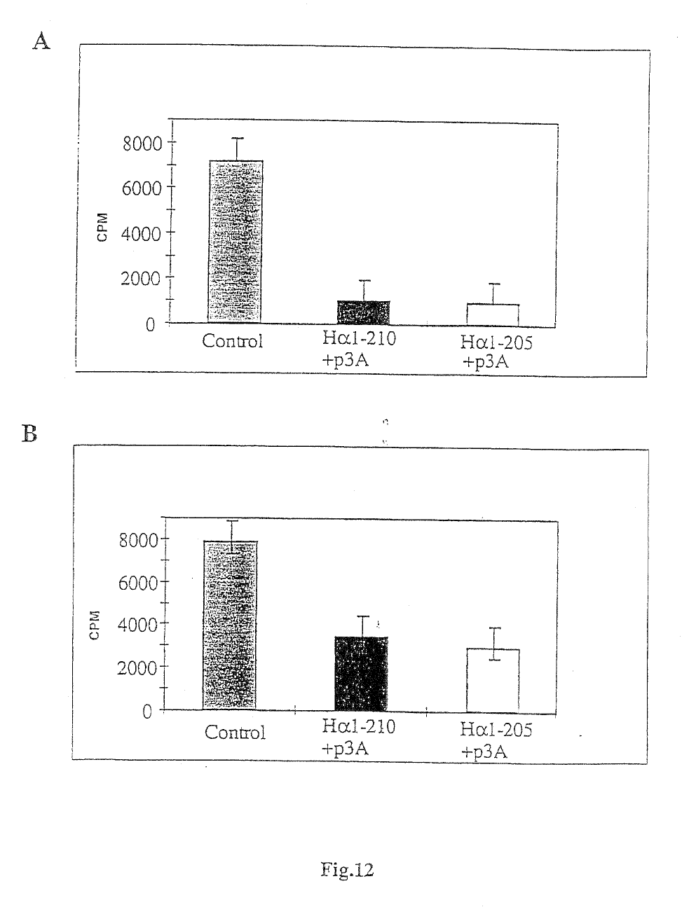

In the second part of the experiment, the potential of oral administration of Hα1-205+p3A to modulate an ongoing disease (in rats immunized with AChR) was investigated. In this experiment, a denatured preparation of Hα1-205+p3A (designated denHα1-215+p3A) was employed for oral treatment of sick rats. Denaturation of Hα1-205+p3A was performed in 6M guanidine HCL, followed by reduction with 0.1M β-mercaptoethanol and carboxymethylation with 0.15M iodoacetamide. Rats with a mild form of EAMG (clinical score of about 1) were pooled and divided randomly into two groups. Rats in the experimental group were fed 7 times with three days interval, each time with 0.3 mg of denHα1-205+p3A per rat, and rats in the control group were fed with ovalbumin. The rats were evaluated weekly for clinical symptoms and for their body weight. As seen in FIG. 13, the disease was arrested in the rats treated orally with the recombinant fragment and their body weight increased. On the other hand the disease progressed in rats of the control group and the rats lost weight gradually.

These protection and suppression effects on EAMG shown in this Example indicate that the polypeptides of the invention affect the autoimmune response to AChR in a manner that may be employed for immunotherapy of myasthenia gravis. Thus, the nasal or oral route of administration could provide a convenient therapeutic modality in humans.

### EXAMPLE 2

The present inventors demonstrated in Example 1 that oral or nasal administration of recombinant fragments of the acetylcholine receptor (AChR) prevents the induction of experimental autoimmune myasthenia gravis (EAMG) and suppresses ongoing EAMG in rats. The present inventors have now studied in the experiments described in this example the role of spatial conformation of these recombinant fragments in determining their tolerogenicity. Two fragments corresponding to the extracellular domain of the human AChR α-subunit and differing in conformation were tested: Hα1-205 expressed with no fusion partner and Hα1-210 fused to thioredoxin (Trx-Hα1-210). The conformational similarity of the fragments to intact AChR was assessed by their reactivity with α-bungarotoxin and with anti-AChR mAbs, specific for conformation-dependent epitopes. Oral administration of the “more native” fragment, Trx-H1α-210, at the acute phase of disease led to exacerbation of EAMG, accompanied by an elevation of AChR-specific humoral and cellular reactivity, increased levels of Th1-type cytokines (IL-2, IL-12), decreased levels of Th2 (IL-10) or Th3 (TGF-β) type cytokines and higher expression of costimulatory factors (CD28, CTLA4, B7-1, B7-2, CD40L and CD40). On the other hand, oral administration of the “less native” fragments Hα1-205 or denatured Trx-Hα1-210, suppressed ongoing EAMG and led to opposite changes in the immunological parameters. These results demonstrate that the native conformation of AChR-derived fragments renders them immunogenic and immunopathogenic and therefore not suitable for treatment of myasthenia gravis. The conformation of tolerogens should therefore be given careful attention when considering oral tolerance for treatment of autoimmune diseases. The experiments and results are presented and discussed below.

### Materials and Methods

Animals

Female Lewis rats (6-7 weeks of age) were purchased from the animal breeding center of the Weizmann Institute of Science (Rehovot, Israel).

Antigen Preparation

AChR was purified from Torpedo californica electric organ by affinity chromatography as previously described (Aharonov et al., 1977). Recombinant fragments were synthesized by PCR on cDNA prepared from total RNA of the human TE671 cell line. The recombinant fragment Hα1-210 containing the P3A exon (Barchan et al., 1998), was expressed as a fusion protein with thioredoxin (Trx-Hα1-210) in pThioHis-A (Invitrogen, USA) or with glutathion S-transferase (GST-Hα1-210) (Barchan et al., 1998) and Hα1-205 was expressed in pET8-C with no fusion partner. All the recombinant proteins, present in inclusion bodies, were solubilized by 9M urea followed by serial dialyses in 50 mM Tris buffer, pH 8.0. Chemical modification, by reduction and carboxymethylation of recombinant fragments, was performed by reduction with 0.1M of 2-ME in 6M guanidine HCl/0.2M Tris buffer, pH 8.8, followed by blocking of the sulfhydryl groups with iodoacetamide as previously described (Bartfeld et al., 1978). The denatured forms of Trx-Hα1-210 or Hα1-205 were designated denTrx-Hα1-210 and denHα1-205, respectively.

Western Blot

Electrophoresis and blotting of recombinant proteins and Torpedo AChR were performed essentially as described (Barchan et al., 1998). The proteins were resolved in 12% polyacrylamide gels and transferred to a nitrocellulose membrane. After blocking with 0.5% hemoglobin in PBS, mAb 198 (10 μg/ml) was added and incubated for 2 h at 37° C. The membrane was washed and then incubated for 1 h at 37° C. with ¹²⁵I-goat-anti-mouse IgG. After washing, the blots were exposed to an X-ray-sensitive film. Binding to α-bungarotoxin (α-BTX) was detected by overlay with ¹²⁵I-α-BTX (2×10⁻⁹M) followed by washing and autoradiography.

Inhibition of mAb 198 Binding to AChR.

Microtiter plates were coated with Torpedo AChR (1 μg/ml) in PBS and incubated overnight at 4° C. After blocking of the plates, mAb 198 preincubated in the presence of different concentrations of recombinant proteins, was added to the wells. Bound mAb 198 was detected by incubation with alkaline phosphatase-conjugated goat anti-rat IgG (1:10,000 dilution), followed by determination of alkaline phosphatase activity.

Induction and Clinical Evaluation of EAMG

Rats were immunized once in both hind foot pads by s.c. injection of Torpedo AChR (45 μg/rat) emulsified in CFA containing additional Mycobacterium tuberculosis (1 mg/rat; Difco Labs, Detroit, Mich.). Clinical severity of EAMG was graded as follows: grade 0, rats with normal muscle strength; grade 1, mildly decreased activity, weak grip, with fatigability; grade 2, weakness, hunched posture at rest, decreased body weight, tremor; 3, severe generalized weakness, marked decrease in body weight, moribund; 4, dead. Animals were evaluated weekly for 7-10 weeks following immunization with Torpedo AChR.

Induction of Oral Tolerance

Feeding with the recombinant fragments was initiated at the acute phase of EAMG, 7-10 days after immunization with Torpedo AChR and continued twice a week until the end of the experiment. The amount of recombinant fragments, and of thioredoxin (Trx) and ovalbumin (OVA, as control), was 600 μg/dose/rat in 1 ml Tris buffer (50 mM, pH 8.0).

Anti-AChR Ab Assay

Antibodies to rat muscle AChR were measured by radioimmunoassay with crude rat muscle extract in which the AChR is specifically labeled by ¹²⁵I-α-BTX (Souroujon et al., 1983). Results are expressed as nmols antibody/L serum.

Lymphocyte Proliferation Assay

Draining lymph node cells (LNC) were cultured (5×10⁵/well) in RPMI 1640 medium supplemented with HEPES, sodium pyruvate, glutamine, 2-ME, antibiotics, nonessential amino acids and 0.5% normal rat serum, either alone or in the presence of Torpedo AChR, Trx-Hα1-210, Hα1-205, or Con A. Proliferation was assessed by measuring (³H)-thymidine (0.5 μCi/well) incorporation during the last 18 h of a 4-day culture period. Results are expressed as Δcpm after subtraction of background of unstimulated cultures from stimulated lymph node cells.

B-Cell Proliferation Assay Based on Alkaline Phosphatase Activity

B-cell proliferation was assayed as described (Hashimoto et al., 1986 and Kasyapa et al., 1992). Draining LNC (1×10⁶/ml) were cultured in the medium used for lymphocyte proliferation supplemented by 10% FCS. The cells were stimulated in vitro with Torpedo AChR (0.01 μg/ml), Trx-Hα1-210 (50 μg/ml), Trx (50 μg/ml), Hα1-205 (50 μg/ml), ConA (2 μg/ml) or LPS (5 μg/ml) in 24-well plates. After 4 days in culture, the cells were harvested, washed and diluted in PBS. For the alkaline phosphatase assay, 100 μl cell suspensions, containing different cell concentrations, were transferred to 96-well plates into which 100 μl/well of substrate solution (p-nitrophenyl phosphate, disodium; 1 mg/ml) was added. The plates were incubated for 2 h at 37° C. in 5% CO₂. The optical density (O.D) at 405 nm was measured and the data are expressed as O.D at 405 nm per number of cells/well.

Determination of Cytokines and Costimulatory Factors

PCR-ELISA was used to assess the levels of mRNA specific for cytokines (IL-2, IL-10, IL-12, IFN-γ and TGF-β) and costimulatory factors (CD40, CD40L, CD28, CTLA4, B7-1 and B7-2). RNA extraction, cDNA synthesis and RT-PCR in the presence of digoxigenin (DIG)-dNTP were performed as described (Zipris et al., 1996) with some modification suggested by the manufacturer of the PCR-ELISA kit (Roche Molecular Biochemicals, Mannheim, Germany).

The sequences of primer pairs specific for rat IL-2, IL-10, IL-12, TGF-β, IFN-γ and β-actin were the same as previously reported (Im et al., 1999). The primer sequences specific for rat costimulatory factors and mouse CD40 are as follows; CD40 sense primer CGCTATGGGGCTGCTTGTTGACAG (SEQ ID NO:15); CD40 antisense primer GACGGTATCAGTGGTCTCAGTGGC (SEQ ID NO:16); CD40 internal primer CAGCCCAGTGGAACAGGGAGATTCGC (SEQ ID NO:17); CD40L sense primer 5′-GATCCTCAAATTGCAGCACA-3′ (SEQ ID NO:18); CD40L antisense primer 5′-AGCCAAAAGATGAGAAGCCA-3′ (SEQ ID NO:19); CD40L internal primer 5′-TGGGAGACAGCTGACGGTTAAAAG-3′ (SEQ ID NO:20); CD28 sense primer 5′-CGGGAATGGGAATTTTACCT-3′ (SEQ ID NO:21); CD28 antisense primer 5′-TCCAGAGCAGTGATGGTGAG-3′ (SEQ ID NO:22); CD28 internal primer 5′-AACATGACACCGCGGAGACTCGGG-3′ (SEQ ID NO:23); CTLA4 sense primer 5′-AGGACTTGGCCTTTTGGAGT-3′ (SEQ ID NO:24); CTLA4 antisense primer 5′-CAGTCCTTGGATGGTGAGGT (SEQ ID NO:25); CTLA4 internal primer 5′-TGATGAGGTCCGGGTGACGGTGCT-3′ (SEQ ID NO:26); B7-1 sense primer 5′-GTGAGAGAAAAGGCATTGCTG-3′ (SEQ ID NO:27); B7-1 antisense primer 5′-GGTTCTTGTTTGTTTCTCTGC-3′ (SEQ ID NO:28); B7-1 internal primer 5′-GGTGCTCTCTGTCATCTCCGGGGT-3′ (SEQ ID NO:29); B7-2 sense primer 5′-GAGGCAAGCTTACTTCAATAGCA-3′ (SEQ ID NO:30); B7-2 antisense primer 5′-ATGCCAGTGTTTCTTGTTTCATT-3′ (SEQ ID NO: 31); B7-2 internal primer 5′-ACACCCACGGGATCAATTATCCTC-3′ (SEQ ID NO:32).

The internal primers were all biotinylated by Biotin-Chem-Link (Roche Molecular Biochemicals, Mannheim, Germany) according to the manufacturer's protocol. The amplified DIG-labeled PCR products were quantified using a PCR-ELISA kit. They were then denatured and hybridized to the suitable cytokine- or costimulatory factor-specific biotinylated internal primers for 3 h at 37° C. with constant shaking. The DIG-labeled PCR product/biotinylated probe hybrids were immobilized on streptavidin-coated 96 well ELISA plates. After washing, the bound PCR products were detected with a peroxidase-conjugated anti-DIG antibody. PCR products were viewed with the peroxidase substrate ABTS, and signals were quantified by absorbance at 405 nm (Tsuruta et al., 1995).

Statistical Analysis

Student's two-tailed T test was used to determine the significance of differences between group means.

### Results

Immunochemical Characterization of AChR-Derived Recombinant Fragments

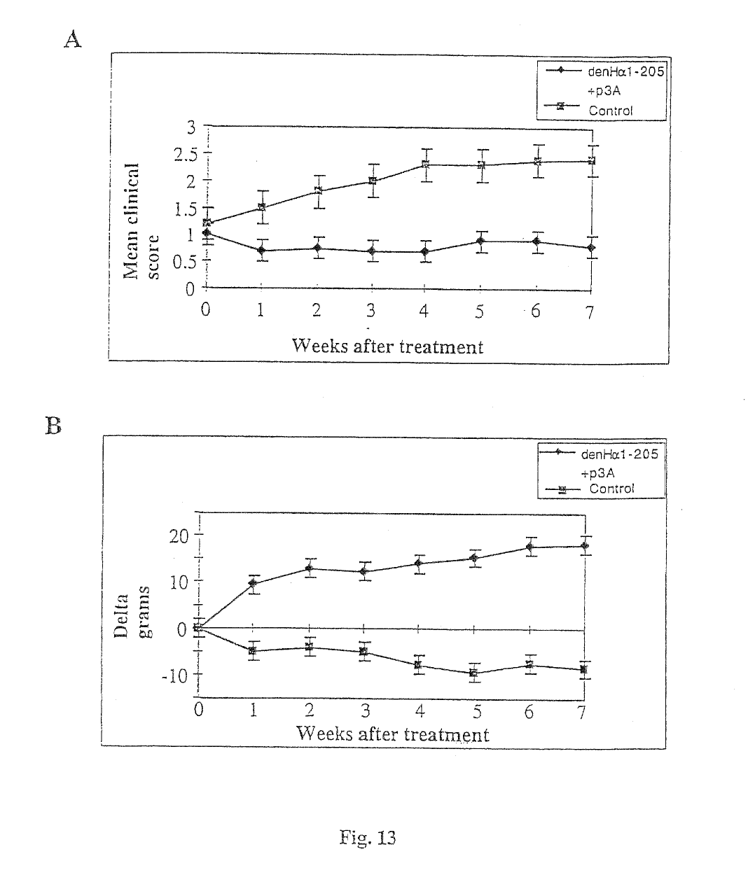

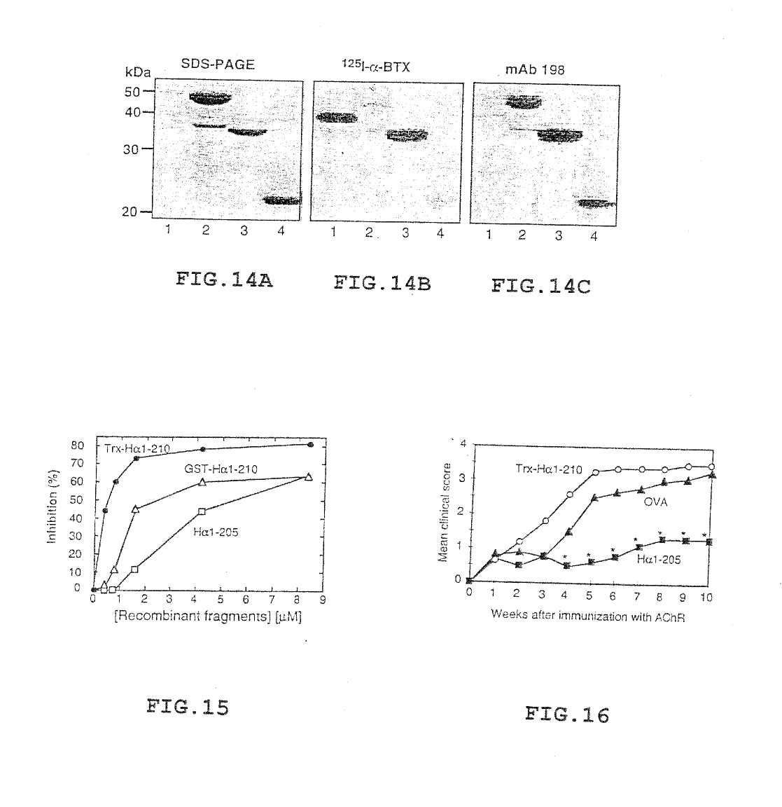

The AChR-derived recombinant fragments of human AChR α-subunit were cloned and expressed either as fusion proteins with thioredoxin (Trx-Hα1-210) or glutathione-S-transferase (GST-Hα1-210), or without a fusion partner (Hα1-205). The extent of their conformational similarity to intact AChR was established by reactivity with α-BTX and mAb 198, an anti-AChR mAb specific for the main immunogenic region (MIR) in the α-subunit which is known to be a conformation-dependent epitope (FIGS. 14B and 14C). As shown in FIG. 14B, Trx-Hα1-210 binds α-BTX to a higher extent than the other two fragments. The weakest α-BTX binder was fragment Hα1-205. Denaturation of Hα1-205 by chemical modification completely abolished its ability to bind α-BTX, assessing the importance of conformation for this binding (data not shown). Similar results were obtained when the blot was overlaid with mAb 5.5 (Mochly-Rosen et al., 1981) which is directed to the acetylcholine binding site (data not shown). The anti-MIR mAb 198 (Tzartos et al., 1981) bound well to Trx-Hα1-210 and, to a lower extent, to the other two fragments (GST-Hα1-210 and Hα1-205) (FIG. 14C).

The binding of the recombinant fragments to mAb 198 in Western blots was correlated with their ability to inhibit the binding of mAb 198 to Torpedo AChR in solution. As shown in FIG. 15, Trx-Hα1-210 inhibited this binding with an IC₅₀ value of 3.0×10⁻⁷ M. The IC₅₀ values for fragments GST-Hα1-210 and Hα1-205 were 1.3×10⁻⁶ M and 3.3×10⁻⁶ M, respectively. In all further experiments, the present inventors focused on two out of the three fragments, which represent the extremes with regard to conformational similarity to intact AChR. Namely, the ‘more native’ fragment, Trx-Hα1-210 and the ‘less native’ fragment, Hα1-205.

Effect of Oral Treatment with Recombinant Fragments on Ongoing EAMG

The role of tolerogen conformation in modulation of EAMG was tested by oral administration of the fragments during the acute phase of disease in rats. The fragments tested were Trx-Hα1-210, Hα1-205 and their respective chemically modified forms, denTrx-Hα1-210 and denHα1-205. OVA and Trx alone were used as controls. Oral administration of the fragments was initiated at the acute phase, 8 days after the induction of EAMG, and was continued twice a week for 9 weeks. Treatment with Trx-Hα1-210 led to aggravation of disease symptoms even as compared with control OVA-treated rats (FIG. 16). In the first five weeks after induction of disease, all rats treated with Trx-Hα1-210, got sick and 6 out of 10 died of EAMG. At that time, 3 out of 100VA-treated rats had died of EAMG whereas Hα1-205-treated rats showed only mild symptoms of EAMG (FIG. 16). Interestingly, oral treatment with the chemically modified, denatured form of Trx-Hα1-210, denTrx-Hα1-210, suppressed EAMG in a similar manner to Hα1-205 (data not shown). Treatment with Trx alone had no effect on EAMG (data not shown), assessing that the fusion partner (Trx) was not responsible for the aggravation of EAMG observed in the Trx-H□1-210-treated rats. By ten weeks after disease induction, 7/10 rats in the Trx-Hα1-210-treated and 6/10 in the OVA-treated group were dead (The mean clinical scores were 3.4 for the Trx-Hα1-210-treated group and 3.2 for the OVA-treated groups). On the other hand, in the Hα1-205-treated group, 3/10 rats were completely healthy and none of the rats died (mean clinical score: 1.3; Table 4).

TABLE 4
Effect of oral treatment with AChR fragments on ongoing EAMG; Acute phase treatment

| | Clinical score^{a} | | | | | Mean | Δ weight | AChR content^{b} | | Anti-rat | T-cell |
|---|---|---|---|---|---|---|---|---|---|---|---|
| | 0 | 1 | 2 | 3 | 4 | clinical | 3-8 weeks | (fmoles/mg | | AChR^{c} | proliferation |
| Trearment | (No/total) | | | | | score^{a} | (gr) | prot.) | (%) | (nM) | (cpm)^{d} |
| OVA | 0/10 | 1/10 | 2/10 | 1/10 | 6/10 | 3.2 | −11 ± 11 | 20.5 ± 3.5 | 45 | 70.5 ± 6.5 | 4241 ± 580 |
| Trx-Hα1-210 | 0/10 | 1/10 | 1/10 | 1/10 | 7/10 | 3.4 | −27 ± 10 | 14.1 ± 3.0 | 31 | 93.5 ± 5.5 | 5205 ± 640 |
| Hα1-205 | 3/10 | 3/10 | 2/10 | 2/10 | 0/10 | 1.3 | 13 ± 10 | 40.1 ± 3.8 | 90 | 31.0 ± 3.5 | 964 ± 250 |

^{a}Evaluated 10 weeks after the induction of EAMG
^{b}Muscle AChR content in normal, age matched rats was 45.5 ± 3.0 fmoles/mg protein and was referred to as the 100% value for this experiment.
^{c}Evaluated 6 weeks after the induction of EAMG and derived from nmoles of α-BTX-binding sites precipitated by one liter of serum.
^{d}In response to Torpedo AChR (0.25 μg/ml)

The evaluation of clinical symptoms of rats treated with different proteins was corroborated by the analyses of muscle AChR content and body weight changes of the rats (Table 4). Rats in the Trx-Hα1-210 and control OVA-treated groups lost 69% and 55% of their muscle AChR content, respectively. In contrast, rats treated by Hα1-205 lost only 10% of their muscle AChR (Table 4). It should be noted that continuous long term oral administration to naive rats (for at least three months) of all tested recombinant fragments has never led to the development of clinical signs of EAMG. However, feeding with the ‘more native’ fragment Trx-Hα1-210 led to elicitation of antibodies to the fragment itself, whereas feeding with Hα1-205 or OVA did not elicit an antibody response to the fed antigen (data not shown).

Oral administration of the fragments was accompanied by different effects on AChR-specific humoral and cellular immune responses. Rats treated orally with Trx-Hα1-210 resulted in an increase in their anti-self AChR antibody levels (93.5±5.5 nM) when compared with the OVA-treated group (70.5±6.5 nM). On the other hand, treatment with Hα1-205 resulted in a decrease in the anti-self AChR antibody level (31.0±3.5 nM). In addition, Trx-Hα1-210-treated rats exhibited also a high AChR-specific proliferative T-cell response, similar to the response in the OVA-treated rats, whereas Hα1-205-treated rats had a suppressed T-cell response (Table 4).

Effect of Tolerogen Conformation on the Expression of Cytokines and Costimulatory Factors

In order to analyze the possible mechanisms underlying the effects that the different fragments exert on EAMG, the levels of cytokines and costimulatory factors were studied in the treated rats. Draining lymph node cells of rats fed with Hα1-205, Trx-Hα1-210 or OVA were removed 5-8 weeks after EAMG induction and cultured for 48 h in the presence of Torpedo AChR. Total RNA was then prepared from the cells and subjected to PCR-ELISA with cytokine-specific or costimulatory factor-specific primers.

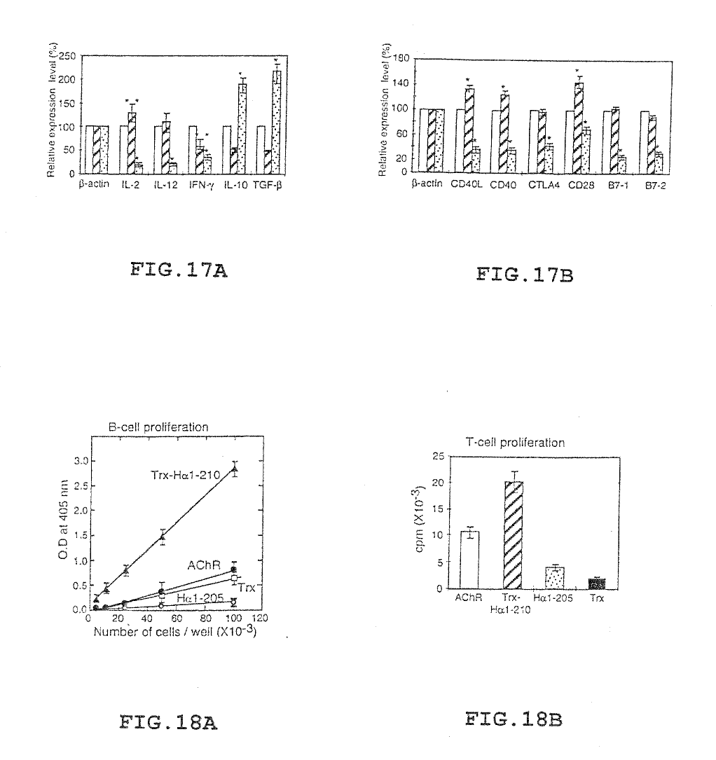

As shown in FIG. 17A, oral treatment with Trx-Hα1-210 resulted in down regulation of IFN-γ, IL-10 and TGF-β and up-regulation in the level of IL-2 (and a slight increase in IL-12) compared with OVA-treated rats. On the other hand, oral treatment with Hα1-205 resulted in suppression of Th1 type (IL-2, IL-12 and IFN-γ) cytokine mRNA levels and in up-regulation of Th2 type (IL-10) or Th3 type (TGF-β) cytokine mRNA levels as already reported by us (Im et al., 1999).

The observed stimulation of AChR-specific T-cell proliferation (Table 4) and up-regulation of Th-1 type cytokine levels (FIG. 17A) suggest alterations in the level of costimulation in Trx-Hα1-210-treated rats. The expression levels of costimulatory factors were tested in the AChR-stimulated LNC which were used for analysis of cytokine levels. As shown in FIG. 17B, oral treatment with Trx-Hα1-210 resulted in up regulation of CD28, CD40 and CD40L compared with OVA-treated rats (p<0.005). Other costimulatory factors such as CTLA4 and B7-1/B7-2 were similarly expressed in Trx-Hα1-210 and OVA-treated rats. On the other hand, oral treatment with the ‘less native’ fragment Hα1-205, which has been an effective tolerogen, resulted in reduced expression levels (p<0.005; as compared to controls) of the costimulatory factors tested, such as CD40L, CD40, CD28, CTLA4 and B7-1/B7-2. This suggests that up-regulated expression of costimulatory factors induced by feeding with Trx-Hα1-210 leads to the increased AChR-specific T-cell proliferation. This activation of autoregulatory T-cells results in up-regulated Th1-type cytokines and down-regulation of Th2 or Th3 cytokines. On the other hand, the protective effect of oral treatment with Hα1-205 is accompanied by down-regulation of costimulatory factor expression, which in turn induces a suppressed AChR-T-cell response.

Effect of Tolerogen Conformation on T and B Cell Proliferation

In order to examine whether the observed upregulation of Th1-type cytokines and of costimulatory factors induced by Trx-H□1-210 feeding, may be also associated with an increased AChR-specific B-cell proliferation, were compared the in vitro response of cells from myasthenic rats to the various fragments. Draining LNC were removed from myasthenic rats (mean clinical score: 2-3) at the chronic stage of disease, 6-8 weeks after EAMG induction. Cells were cultured for 4 days in the presence of Torpedo AChR, Trx-Hα1-210, Hα1-205, Trx, Con A or LPS and the level of B-cell proliferation was determined by alkaline phosphatase activity (which is known to be specific for activated B-cells; Hashimoto et al., 1986 and Kasyapa et al., 1992). Trx-Hα1-210 induced the highest B-cell proliferative response (FIG. 18A), whereas Trx alone had only a minor effect on B-cell proliferation. LPS induced a strong response and ConA did not induce any B-cell proliferative response (data not shown), as expected for activated B-cells. Interestingly, Torpedo AChR induced a lower B-cell proliferation than Trx-Hα1-210, which may be due to its processing in vitro.

T-cell proliferation was also assessed in the same LNC. As shown in FIG. 18B, T-cell proliferation in the presence of Trx-Hα1-210 was higher than in the presence of the other fragments. Trx alone induced only a minor T-cell proliferation (data not shown). The different T-cell responses induced by the two fragments (Trx-Hα1-210 and Hα1-205), may reflect differences in their antigen processing and presentation in the LNC of myasthenic rats.

### Discussion

This example focuses on the role of conformation of orally administered AChR fragments in the induction of systemic suppression of EAMG. Insight was gained on the immunological pathways that follow the oral administration of conformationally different AChR fragments and this also suggest clues to predict what is required from a fed protein to serve as a successful tolerogen.

Rats were fed at the acute phase of EAMG with recombinant fragments, all corresponding to the extracellular domain of the human AChR α-subunit, but differing in their spatial conformation. One of the fragments, Hα1-205 was previously shown by the laboratory of the present inventors to suppress EAMG in rats when administered orally either at the acute or at the chronic phase of disease (Im et al., 1999). The other recombinant fragment, Trx-Hα1-210 corresponds to the same region in the human AChR α-subunit but in contrast to Hα1-205, its 3-D structure is more similar to that of the corresponding region in native intact AChR. This was assessed by its reactivity with α-BTX, mAb 5.5 and mAb 198, all of which are known to recognize conformation-dependent epitopes of AChR. Another recombinant fragment consisting of the same sequence joined to GST (GST-Hα1-210) had intermediate characteristics. The present inventors have demonstrated that in contrast to Hα1-205 that suppresses EAMG, the ‘more native’ fragment, Trx-Hα1-210, fails to do so.

The next goal was to analyze the immunological events that follow the oral administration of these conformationally different fragments, and that result in one case in suppression and in the other case in exacerbation of an existing disease. The present inventors demonstrate that whereas the ‘less native’ fragment, Hα1-205 leads to a decreased humoral and cellular AChR-specific response accompanied by a decrease in the production of pro-inflammatory cytokines and costimulatory factors, the oral administration of the ‘more native’, Trx-Hα1-210 fragment leads to opposite changes. Namely, feeding with Trx-Hα1-210 leads to an elevated AChR-specific humoral and cellular reactivity and to an upregulation of the pro-inflammatory cytokine IL-2 and costimulatory factors accompanied by down-regulation of anti-inflammatory cytokines. Although Trx has been shown to act as a potent chemoattractant and inducer of cytokines (Schenk et al., 1996 and Bertini et al., 1999), the latter effects cannot be attributed to Trx since denatured Trx-Hα1-210 and Trx alone did not act like Trx-Hα1-210.

Previous reports have demonstrated the involvement of the pro-inflammatory cytokines IL-12 and IFN-γ in the induction of EAMG (Balasa et al., 1997; Zhang et al., 1998 and Moiola et al., 1998) and the protective effects of anti-inflammatory cytokines such as IL-10 and TGF-β in autoimmune diseases including EAMG (Xiao et al., 1997). Therefore our observations on the different changes in the cytokine profile following the administration of Hα1-205 and Trx-Hα1-210, may explain the different effects of these two fragments on the course of EAMG.

The opposite consequences of oral administration of fragments differing in their conformation may stem from the repertoire of T and B cell epitopes they are bearing. The ‘more native’ fragment, Trx-Hα1-210 may be recognized by autoreactive B cells already existing in the myasthenic rats, that could serve as antigen-presenting cells required for T-cell activation, as has been implied in other autoimmune diseases (Falcone et al., 1998). Such a fragment is more likely to have deleterious effects upon oral ingestion. The ‘less native’ fragment, Hα1-205, probably bears significantly less, or no pathogenic B-cell epitopes at all, and would therefore not stimulate B-cell proliferation that would in turn lead to AChR-specific T-cell activation. Our B-cell proliferation assay indeed demonstrates that Trx-Hα1-210 can stimulate B cells from sensitized rats whereas Hα1-205, denatured Trx-Hα1-210 and Trx alone, do not. Moreover, oral administration of Trx-Hα1-210 leads to increased levels of CD40L, which is expressed on activated T cells and is known to be an important costimulatory factor in B-cell activation. This factor has also been shown to be essential for AChR-specific immune responses since CD40L-deficient mice (CD40L -/-) are resistant to EAMG induction (Shi et al., 1998). The B-cell activation following the administration of a native AChR fragment could lead to the elevated AChR specific T-cell proliferation (Table 4) and to the observed shift in the cytokine profile from the desired Th2/Th3 response to the myasthenogenic Th1-regulated AChR-specific response. Conversely, when a less native AChR fragment, such as Hα1-205, is orally administered, the level of costimulation is too low to stimulate T-cell activation thus leading to a shift in the cytokine profile in favor of the anti-inflammatory Th2/Th3 cytokines.

In the present study, the present inventors have attempted to induce tolerance when EAMG already exists. In our experimental model, native conformation of the tolerogen employed was not beneficial for the induction of oral tolerance. This might be due to some residual pathogenicity which may result in stimulation of already activated B cells, especially in the case of a highly immunogenic autoantigen as AChR. It is therefore important to delineate the requirements for an effective tolerogen. In the case of EAMG, the present inventors believe that myasthenogenicity of the tested fragments upon active immunization provides one such clue. Injections of large amounts of Trx-Hα1-210 (500 μg/dose in CFA) was observed to result in clinical signs of EAMG, while injection of the same dose of Hα1-205 was observed to result only in a transient disease characterized by very mild symptoms (mean clinical score: 1). Nevertheless, it should be stressed that even long term oral administration of any of the tested fragments never led to clinical signs of EAMG. Another clue is based on the ability to elicit a humoral response to the fed fragment. Oral feeding with the ‘more native’ fragment Trx-Hα1-210 led to production of anti-fragment antibodies, whereas feeding with denTrx-Hα1-210 or Hα1-205 did not elicit any humoral response.

The molecular features required for immunopathogenicity and tolerogenicity may be distinct from each other, and there is an advantage to be able to control them as desired. This distinction may be particularly important for attempts to induce tolerance in an already existing disease. So far, most of the oral tolerance studies in experimental autoimmune diseases describe prevention experiments in which the tolerogen was introduced prior to disease induction, when antigen-specific activated B or T cells still do not exist. It may therefore be somewhat misleading to design clinical trials on the basis of such prevention studies. Moreover, this may be one of the reasons why clinical trials on ongoing human autoimmune diseases have not been very successful.

In conclusion, this study suggests that the spatial conformation of an orally administered tolerogen should be given careful attention when considering oral treatment for the induction of systemic tolerance in established antibody-mediated autoimmune diseases such as myasthenia gravis.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Aharonov, A., Abramsky, O., Tarrab-Hazdai, R. and Fuchs, S. (1975). Humoral antibodies to acetylcholine receptor in patients with myasthenia gravis. Lancet 2: 340.
Aharonov, A., R. Tarrab-Hazdai, I. Silman, and S. Fuchs. (1977). Immunochemical studies on acetylcholine receptor fraction from Torpedo californica. Immunochemistry 14:129.
Asher, O., Kues, W. A., Witzemann, V., Tzartos, S. J., Fuchs, S, and Souroujon, M. C. (1993). Increased gene expression of acetylcholine receptor and myogenic factors in passively transferred experimental autoimmune myasthenia gravis. J Immunol 151: 6442-50.
Asher, O., Neumann, D. and Fuchs, S. (1988). Increased levels of acetylcholine receptor α-subunit mRNA in experimental autoimmune myasthenia gravis. FEBS Lett 233: 277-81.
Ausubel et al., eds., (1993-1998) “Current Protocols in Molecular Biology” in Current Protocols autoimmune diseases. Biopolymers 43:323.
Balasa, B., C. Deng, J. Lee, L. M. Bradley, D. K. Dalton, P. Christadoss, and N. Sarvetnick. (1997). Interferon gamma (IFN-gamma) is necessary for the genesis of acetylcholine receptor-induced clinical experimental autoimmune myasthenia gravis in mice. J Exp Med 186:385.
Balass, M., Heldman, Y., Cabilly, S., Givol, D., Katchalski, K. E. and Fuchs, S. (1993). Identification of a hexapeptide that mimics a conformation-dependent binding site of acetylcholine receptor by use of a phage-epitope library. Proc Natl Acad Sci USA 90: 10638-42.
Barchan, D., Kachalsky, S., Neumann, D., Vogel, Z., Ovadia, M., Kochba, E. and Fuchs, S. (1992). How does the mongoose fight the snake: the binding site of the mongoose acetylcholine receptor. Proc. Nat. Acad. Sci. USA 89: 7717-7721.
Barchan, D., M. C. Souroujon, S. H. Im, C. Antozzi, and S. Fuchs. (1999). Antigen-specific modulation of experimental myasthenia gravis: Nasal tolerization with recombinant fragments of the human acetylcholine receptor alpha-subunit. Proc Natl Acad Sci USA 96:8086.
Barchan, D., O. Asher, S. J. Tzartos, S. Fuchs, and M. C. Souroujon. (1998). Modulation of the anti-acetylcholine receptor response and experimental autoimmune myasthenia gravis by recombinant fragments of the acetylcholine receptor. Eur J Immunol 28:616.
Bartfeld, D. and Fuchs, S. (1978). Specific immunosupression of experimental autoimmune myasthenia gravis by denatured acetylcholine receptor. Proc. Natl. Acad. Sci. USA. 75: 4006-4010.
Bartfeld, D., and S. Fuchs. (1978). Specific immunosupression of experimental autoimmune myasthenia gravis by denatured acetylcholine receptor. Proc Natl. Acad Sci USA. 75:4006.
Beeson, D., Morris, A., Vincent, A. and Newson-Davis, J. (1990). The human muscle nicotinic acetylcholine receptor α-subunit exists as two isoforms: a novel exon. EMBO J. 9: 2101-2106.
Bergerot, I., N. Fabien, A. Mayer, and C. Thivolet. (1996). Active suppression of diabetes after oral administration of insulin is determined by antigen dosage. Ann N Y Acad Sci 778:362.
Bertini, R., O. M. Howard, H. F. Dong, J. J. Oppenheim, C. Bizzarri, R. Sergi, G. Caselli, S. Pagliei, B. Romines, J. A. Wilshire, M. Mengozzi, H. Nakamura, J. Yodoi, K. Pekkari, R. Gurunath, A. Holmgren, L. A. Herzenberg, and P. Ghezzi. (1999). Thioredoxin, a redox enzyme released in infection and inflammation, is a unique chemoattractant for neutrophils, monocytes, and T cells. J Exp Med 189:1783.
Changeux, J. P., Devillers-Thiery, A. and Chemouilli, P. (1984). The Acetylcholine receptor: an allosteric protein. Science. 225: 1335-1345.
Dick, A. D., Cheng, Y. F., McKinnon, A., Liversidge, J. and Forrester, J. V. (1993). Nasal administration of retinal antigens suppresses the inflammatory response in experimental allergic uveoretinitis. A preliminary report of intranasal induction of tolerance with retinal antigens. Br J Opthalmol 77: 171-5.
Drachman, D. B. (1994). Myasthenia gravis. N Engl J Med 330: 1797-810.
Drachman, D. B. (1996). Immunotherapy in neuromuscular disorders:current and future strategies. Muscle & Nerve 19:1239.
Ermak, T. H., H. R. Bhagat, and J. Pappo. (1994). Lymphocyte compartments in antigen-sampling regions of rabbit mucosal lymphoid organs. Arm J Trop Med Hyg 50:14.
Falcone, M., J. Lee, G. Patstone, B. Yeung, and N. Sarvetnick. (1998). B lymphocytes are crucial antigen-presenting cells in the pathogenic autoimmune response to GAD65 antigen in nonobese diabetic mice. J Immunol 161:1163.
Fowler, E., and H. L. Weiner. (1997). Oral tolerance: elucidation of mechanisms and application to treatment of diseases. Biopolymers 43:323.
Friedman, A., and H. Weiner. (1994). Induction of anergy or active suppression following oral tolerance is determined by antigen dosage. Proc Natl Acad Sci USA 91:6688.
Gregerson, D. S., W. F. Obritsch, and L. A. Donoso. (1993). Oral tolerance in experimental autoimmune uveoretinitis. Distinct mechanisms of resistance are induced by low dose vs high dose feeding protocols. J Immunol 151:5751.
Hashimoto, N., and R. H. Zubler. (1986). Colorimetric B cell proliferation assay based on alkaline phosphatase activity. Selective measurement of B cell proliferation in the presence of other cell types. J Immunol Methods 90:97.
Im, S. H., D. Barchan, S. Fuchs, and M. C. Souroujon. (1999). Suppression of ongoing experimental myasthenia by oral treatment with an acetylcholine receptor recombinant fragment. J Clin Invest 104:1723
Karlin, A. (1980) Molecular properties of the acetylcholine receptors. in In The Cell Surface and Neuronal Function. G. Poste, C. W. Cotman and G. L. Nicolson (eds), 191-260.
Kasyapa, C. S., and M. Ramanadham. (1992). Alkaline phosphatase activity is expressed only in B lymphocytes committed to proliferation [published erratum appears in Immunol Lett 1992 August; 33(3):315]. Immunol Lett 31:111.
Lennon, V. A., Lambert, E. H., Leiby, K. R., Okarma, T. B. and Talib, S. (1991). Recombinant human acetylcholine receptor α-subunit induces chronic experimental autoimmune myasthenia gravis. J. Immunol. 146, 2245-2248.
Li, H. L., F. D. Shi, X. F. Bai, Y. M. Huang, P. H. van der Meide, B. G. Xiao, and H. Link. (1998). Nasal tolerance to experimental autoimmune myasthenia gravis: tolerance reversal by nasal administration of minute amounts of interferon-gamma. Clin Immunol Immunopathol 87:15.
Lindstrom, J. M., Einarson, B. L., Lennon, V. A. and Seybold, M. E. (1976). Pathological mechanisms in experimental autoimmune myasthenia gravis. I. Immunogenicity of syngeneic muscle acetylcholine receptor and quantitative extraction of receptor and antibody-receptor complexes from muscles of rats with experimental autoimmune myasthenia gravis. J. Exp. Med. 144, 726-738.
Lindstrom, J. M., Engel, A. G., Seybold, M. E., Lennon, V. A. and Lambert, E. H. (1976a). Pathological mechanisms in experimental autoimmune myasthenia gravis. II. Passive transfer of experimental autoimmune myasthenia gravis in rats with anti-acetylcholine receptor antibodies. J. Exp. Med. 144, 739-753.
Loutrari, H., Kokla, A. and Tzartos, S. J. (1992). Passive transfer of experimental myasthenia gravis via antigenic modulation of acetylcholine receptor. Eur J Immunol 22: 2449-52.
Loutrari, H., Tzartos, S. J. and Claudio, T. (1992a). Use of Torpedo-mouse hybrid acetylcholine receptors reveals immunodominance of the alpha subunit in myasthenia gravis antisera. Eur J Immunol 22: 2949-56.
Ma, C. G., Zhang, G. X., Xiao, B. G., Link, J., Olsson, T. and Link, H. (1995). Suppression of experimental autoimmune myasthenia gravis by nasal administration of acetylcholine receptor. J Neuroimmunol 58: 51-60.
McGhee, J. R., J. Mestecky, M. T. Dertzbaugh, J. H. Eldridge, M. Hirasawa, and H. Kiyono. (1992). The mucosal immune system: from fundamental concepts to vaccine development. Vaccine 10:75.
Meinkoth et al., Anal. Biochem. 138:267-284 (1984)
Mochly-Rosen, D. and Fuchs, S. (1981). Monoclonal anti-acetylcholine receptor directed against the cholinergic binding site. Biochemistry 20: 5920-5924.
Mochly-Rosen, D., and S. Fuchs. (1981). Monoclonal anti-acetylcholine receptor directed against the cholinergic binding site. Biochemistry 20:5920.
Moiola, L., F. Galbiati, G. Martino, S. Amadio, E. Brambilla, G. Comi, A. Vincent, L. M. Grimaldi, and L. Adorini. (1998). IL-12 is involved in the induction of experimental autoimmune myasthenia gravis, an antibody-mediated disease. Eur J Immunol 28:2487.
Nagler-Anderson, C., L. A. Bober, M. E. Robinson, G. W. Siskind, and G. J. Thorbecke. (1986). Suppression of type II collagen-induced arthritis by intragastric administration of soluble type II collagen. Proc Natl Acad Sci USA 83:7443.
Neumann, D., Gershoni, J. M., Fridkin, M. and Fuchs, S. (1985). Antibodies to synthetic peptides as probes for the binding site on the alpha subunit of the acetylcholine receptor. Proc Natl Acad Sci USA 82: 3490-3.
Noda, M., Furutani, Y., Takahashi, H., Toyosato, M., Tanabe, T., Shimizu, S., Kikyotani, S., Kayano, T., Hirose, T., Inayama, S, and Numa, S. (1983). Cloning and sequence analysis of calf cDNA and human genomic DNA encoding α-subunit precursor of muscle acetylcholine receptor. Nature 305, 818-823.
Nussenblatt, R. B., S. M. Whitcup, M. D. de Smet, R. R. Caspi, A. T. Kozhich, H. L. Weiner, B. Vistica, and I. Gery. (1996). Intraocular inflammatory disease (uveitis) and the use of oral tolerance: a status report. Ann N Y Acad Sci 778:325.
Patrick, J. and Lindstrom, J. M. (1973). Autoimmune response to acetylcholine receptor. Science 180: 871-872.
Sambrook et al., eds. (1989) “Molecular Cloning: A Laboratory Manual”, 2nd ed., Cold Spring Harbor Press.
Schatz, D. A., D. G. Rogers, and B. H. Brouhard. (1996).

Prevention of insulin-dependent diabetes mellitus: an overview of three trials. Cleve Clin J Med 63:270.
Schenk, H., M. Vogt, W. Droge, and K. Schulze-Osthoff. (1996). Thioredoxin as a potent costimulus of cytokine expression. J Immunol 156:765.
Schoepfer, R., Luther, M. and Lindstrom, J. (1988). The human medulloblastoma cell line TE671 expresses a muscle-like acetylcholine receptor. Cloning of the alpha-subunit cDNA. FEBS Lett 226: 235-40.
Shi, F. D., B. He, H. Li, D. Matusevicius, H. Link, and H. G. Ljunggren. (1998). Differential requirements for CD28 and CD40 ligand in the induction of experimental autoimmune myasthenia gravis. Eur J Immunol 28:3587.
Shi, F. D., X. F. Bai, H. L. Li, Y. M. Huang, P. H. Van der Meide, and H. Link. (1998). Nasal tolerance in experimental autoimmune myasthenia gravis (EAMG): induction of protective tolerance in primed animals. Clin Exp Immunol 111:506.
Sieper, J., S. Kary, H. Sorensen, R. Alten, U. Eggens, W. Huge, F. Hiepe, A. Kuhne, J. Listing, N. Ulbrich, J. Braun, A. Zink, and N. A. Mitchison. (1996). Oral type II collagen treatment in early rheumatoid arthritis. A double-blind, placebo-controlled, randomized trial. Arthritis Rheum 39:41.
Smith, D. B. and Johnson, K. S. (1988). Single-step purification of polypeptides expressed in Escherichia coli as fusions with glutathione S-transferase. Gene 67: 31-40.
Sophianos, D. and Tzartos, S. J. (1989). Fab fragments of monoclonal antibodies protect the human acetylcholine receptor against antigenic modulation caused by myasthenic sera. J. Autoimmunity 2, 777-789.
Souroujon, M. C., Carmon, S. and Fuchs, S. (1992). Modulation of anti-acetylcholine receptor antibody specificities and of experimental autoimmune myasthenia gravis by synthetic peptides. Immunol Lett 34: 19-25.
Souroujon, M. C., Carmon, S. and Fuchs, S. (1993). Regulation of experimental autoimmune myasthenia gravis by synthetic peptides of the acetylcholine receptor. Ann N Y Acad Sci 681: 332-334.
Souroujon, M. C., D. Mochly-Rosen, A. S. Gordon, and S. Fuchs. (1983). Interaction of monoclonal antibodies to Torpedo acetylcholine receptor with the receptor of skeletal muscle. Muscle and Nerve 6:303.
Souroujon, M. C., Pachner, A. R. and Fuchs, S. (1986). The treatment of passively transferred experimental myasthenia with anti-idiotypic antibodies. Neurology 36: 622-5.
Souroujon, M. C., Pizzighella, S. Mochly-Rosen, D. and Fuchs, S. (1985). Antigenic specificity of acetylcholine receptor in developing muscle: Studies with monoclonal antibodies. J. of Neuroimmunology, 8; 159-166.
Terato, K., X. J. Ye, H. Miyahara, M. A. Cremer, and M. M. Griffiths. (1996). Induction by chronic autoimmune arthritis in DBA/1 mice by oral administration of type II collagen and Escherichia coli lipopolysaccharide. Br J Rheumatol 35:828.
Thompson, H. S., and N. A. Staines. (1986). Gastric administration of type II collagen delays the onset and severity of collagen-induced arthritis in rats. Clin Exp Immunol 64:581.
Trentham, D. E., R. A. Dynesius-Trentham, E. J. Orav, D. Combitchi, C. Lorenzo, K. L. Sewell, D. A. Hafler, and H. L. Weiner. (1993). Effects of oral administration of type II collagen on rheumatoid arthritis. Science 261:1727.
Tsuruta, H., S. Matsui, K. Oka, T. Namba, M. Shinngu, and M. Nakamura. (1995). Quantitation of IL-1 beta mRNA by a combined method of RT-PCR and an ELISA based on ion-sensitive field effect transistor. J Immunol Methods 180:259.
Tzartos, S. and Lindstrom, J. (1980). Monoclonal antibodies used to probe acetylcholine receptor structure: localization of the main immunogenic region and detection of similarities between subunits. Proc. Natl. Acad. Sci. USA 77: 755.
Tzartos, S. J., D. E. Rand, B. L. Einarson, and J. M. Lindstrom. (1981). Mapping of surface structures of electrophorus acetylcholine receptor using monoclonal antibodies. J Biol Chem 256.
Tzartos, S. J., Hochschwender, S., Vasquez, P. and Lindstrom, J. (1987). Passive transfer of experimental autoimmune myasthenia gravis by monoclonal antibodies to the main immunogenic region of the acetylcholine receptor. J. Neuroimmunol. 15: 185-194.
Weiner, H. L. (1997). Oral tolerance for the treatment of autoimmune diseases. Annu Rev Med 48:341.
Weiner, H. L., Friedman, A., Miller, A., Khoury, S. J., al, S. A., Santos, L., Sayegh, M., N, u. R., Trentham, D. E. and Hafler, D. A. (1994). Oral tolerance: immunologic mechanisms and treatment of animal and human organ-specific autoimmune diseases by oral administration of autoantigens. Annu Rev Immunol 12: 809-837.
Weiner, H. L., G. A. Mackin, M. Matsui, E. J. Orav, S. J. Khoury, D. M. Dawson, and D. A. Hafler. (1993). Double-blind pilot trial of oral tolerization with myelin antigens in multiple sclerosis. Science 259:1321.
Whitacre, C. C., I. E. Gienapp, C. G. Orosz, and D. M. Bitar. (1991). Oral tolerance in experimental autoimmune encephalomyelitis. III. Evidence for clonal anergy. J Immunol 147:2155.
Wilson, P. T., Lentz, T. L. and Hawrot, E. (1985). Determination of the primary amino acid sequence specifying the α-bungarotoxin binding site on the α-subunit of the acetylcholine receptor from Torpedo californica. Proc. Natl. Acad. Sci. USA. 82: 8790-8794.

Xiao, B. G., and H. Link. (1997). Mucosal tolerance: a two-edged sword to prevent and treat autoimmune diseases. Clin Immunol Immunopathol 85:119.
Zhang, G. X., B. G. Xiao, X. F. Bai, A. Orn, P. H. van der Meide, and H. Link. (1998). IFN-gamma is required to induce experimental autoimmune myasthenia gravis. Ann N Y Acad Sci 841:576.
Zipris, D., D. L. Greiner, S. Malkani, B. Whalen, J. P. Mordes, and A. A. Rossini. (1996). Cytokine gene expression in islets and thyroids of BB rats. IFN-gamma and IL-12p40 mRNA increase with age in both diabetic and insulin-treated nondiabetic BB rats. J Immunol 156:1315.

What is claimed is:

1. An isolated DNA molecule consisting of a sequence coding for a polypeptide tolerogen which suppresses the autoimmune response of an individual to acetylcholine receptor, wherein said polypeptide tolerogen is selected from the group consisting of:
(i) a polypeptide consisting of amino acid residues 1-121 of SEQ ID NO:2;
(ii) a polypeptide consisting of amino acid residues 122-210 of SEQ ID NO:2;
(iii) a polypeptide Hα1-205 consisting of amino acid residues 1-205 of SEQ ID NO:2;
(iv) a polypeptide comprising the amino acid sequence of SEQ ID NO:6;
(v) a polypeptide comprising amino acid residues 1-146 of SEQ ID NO:6;
(vi) a polypeptide comprising the amino acid sequence of SEQ ID NO:8;
(vii) a polypeptide comprising an amino acid sequence with at least 95% sequence identity to the amino acid sequence of SEQ ID NO:6, amino acid residues 1-146 of SEQ ID NO:6, or the amino acid sequence of SEQ ID NO:8;
(viii) a fragment of (i) comprising amino acid residues 61-76 of SEQ ID NO:2;
(ix) a fragment of (ii) comprising amino acid residues 184-210 of SEQ ID NO:2;
(x) a polypeptide as defined in (i)-(iii), fused to an additional polypeptide at its N- and/or C-termini, wherein a human acetylcholine receptor α-subunit portion, consisting of amino acid residues 1-121 of SEQ ID NO:2, amino acid residues 122-210 of SEQ ID NO:2, or amino acid residues 1-205 of SEQ ID NO:2, of said fused polypeptide does not assume the native conformation of the α subunit of the human acetylcholine receptor as determined from a binding assay to α-bungarotoxin, where weaker binding to α-bungarotoxin when compared to the acetylcholine receptor (AchR) α-subunit extracellular domain indicates said fused polypeptide has not assumed the native conformation of the α-subunit of AchR; and
(xi) a fragment as defined in (viii) or (ix) fused to an additional polypeptide at its N- and/or C-termini,
with the proviso that said polypeptide tolerogen does not consist of a sequence consisting of residues 1-210 of SEQ ID NO:2 and with the proviso that said polypeptide tolerogen does not consist of residues 1-210 of SEQ ID NO:2 and one additional residue.

2. The isolated DNA molecule of claim 1, wherein said polypeptide tolerogen consists of amino acid residues 1-121 of SEQ ID NO:2.

3. The isolated DNA molecule of claim 1, wherein said polypeptide tolerogen consists of amino acid residues 122-210 of SEQ ID NO:2.

4. The isolated DNA molecule of claim 1, wherein said polypeptide tolerogen consists of amino acid residues 1-205 of SEQ ID NO:2.

5. The isolated DNA molecule of claim 1, wherein said polypeptide tolerogen comprises the amino acid sequence of SEQ ID NO:6.

6. The isolated DNA molecule of claim 1, wherein said polypeptide tolerogen comprises amino acid residues 1-146 of SEQ ID NO:6.

7. The isolated DNA molecule of claim 1, wherein said polypeptide tolerogen comprises the amino acid sequence of SEQ ID NO:8.

8. The isolated DNA molecule claim 1, wherein said polypeptide tolerogen comprises an amino acid sequence with at least 95% sequence identity to the amino acid sequence of SEQ ID NO:6, amino acid residues 1-146 of SEQ ID NO:6, or the amino acid sequence of SEQ ID NO:8.

9. The isolated DNA molecule of claim 1, wherein said polypeptide tolerogen is a fragment of (i) comprising amino acid residues 61-76 of SEQ ID NO:2.

10. The isolated DNA molecule of claim 1, wherein said polypeptide tolerogen is a fragment of (ii) comprising amino acid residues 184-210 of SEQ ID NO:2.

11. The isolated DNA molecule of claim 1, wherein said polypeptide tolerogen is said fusion polypeptide as defined in (x).

12. The isolated DNA molecule of claim 11, wherein said additional polypeptide is glutathione S-transferase.

13. The isolated DNA molecule of claim 1, wherein said polypeptide tolerogen is a fragment as defined in (viii) or (ix), fused to an additional polypeptide at its N- and/or C-termini.

14. The isolated DNA molecule of claim 1, which is selected from the group consisting of:
(i) a DNA molecule consisting of nucleotides 1 to 363 of SEQ ID NO:1;
(ii) a DNA molecule consisting of nucleotides 364 to 630 of SEQ ID NO:1;
(iii) a DNA molecule consisting of nucleotides 1 to 615 of SEQ ID NO:1;
(iv) a DNA molecule comprising the nucleotide sequence of SEQ ID NO:5;
(v) a DNA molecule comprising nucleotides 1 to 438 of SEQ ID NO:5;
(vi) a DNA molecule comprising the nucleotide sequence of SEQ ID NO:7; and
(vii) a DNA molecule which codes for a polypeptide encoded by the DNA sequence of (i), (ii), (iii), (iv), (v) or (vi).

15. The isolated DNA molecule of claim 14, which consists of nucleotides 1 to 363 of SEQ ID NO:1.

16. The isolated DNA molecule of claim 14, which consists of nucleotides 364 to 630 of SEQ ID NO:1.

17. The isolated DNA molecule of claim 14, which consists of nucleotides 1 to 615 of SEQ ID NO:1.

18. The isolated DNA molecule of claim 14, which comprises the nucleotide sequence of SEQ ID NO:5.

19. The isolated DNA molecule of claim 14, which comprises nucleotides 1 to 438 of SEQ ID NO:5.

20. The isolated DNA molecule of claim 14, which comprises the nucleotide sequence of SEQ ID NO:7.

21. A replicable expression vector comprising the DNA molecule of claim 14.

22. An isolated prokaryotic or isolated eukaryotic host cell transformed with the replicable expression vector of claim 21.

23. A process for preparing a polypeptide which suppresses the autoimmune response of an individual to acetylcholine receptor, comprising:
(i) culturing the transformed host cell of claim 22 under conditions promoting expression of the polypeptide to express the polypeptide; and
(ii) isolating the expressed polypeptide.

24. An isolated DNA molecule coding for a polypeptide tolerogen which suppresses the autoimmune response of an individual to acetylcholine receptor, wherein said polypeptide tolerogen is either (a) a polypeptide consisting of amino acid residues 1-121 of SEQ ID NO:2 fused to an additional polypeptide at its N- and/or C-termini, (b) a polypeptide consisting of amino acid residues 1-205 fused to an additional polypeptide at its N- and/or C-termini, or (c) a polypeptide Hα1-210 consisting of amino acid residues 1-210 of SEQ ID NO:2 fused to an additional polypeptide at its N- and/or C-termini, wherein a human acetylcholine receptor α-subunit portion, consisting of amino acid residues 1-121 of SEQ ID NO:2, amino acid residues 1-205 of SEQ ID NO:2, or amino acid residues 1-210 of SEQ ID NO:2, of said fused polypeptide does not assume the native conformation of the α-subunit of the human acetylcholine receptor as determined from a binding assay to α-bungarotoxin, where weaker binding to α-bungarotoxin when compared to the corresponding portion from the acetylcholine receptor (AchR) α-subunit extracellular domain indicates said fused polypeptide has not assumed the native conformation of the α-subunit of AchR, with the proviso that said polypeptide tolerogen does not consist of a sequence consisting of residues 1-210 of SEQ ID NO:2 and with the proviso that said polypeptide tolerogen does not consist of residues 1-210 of SEQ ID NO:2 and one additional residue.

25. The isolated DNA molecule of claim 24, wherein said additional polypeptide is glutathione S-transferase (GST) and is fused to the human acetylcholine receptor α subunit portion at its N- and/or C-termini.

26. A replicable expression vector comprising the DNA molecule of to claim 24.

27. An isolated prokaryotic or isolated eukaryotic host cell transformed with the replicable expression vector of claim 26.

28. A process for preparing a polypeptide which suppresses the autoimmune response of an individual to acetylcholine receptor, comprising:
(i) culturing the transformed host cell of claim 27 under conditions promoting expression of the polypeptide to express the polypeptide; and
(ii) isolating the expressed polypeptide.
